(19) United States
(12) Patent Application Publication
(10) Pub. No.: US 2024/0324913 A1
(43) Pub. Date: Oct. 3, 2024

(54) NON-ENZYMATIC POTASSIUM SENSOR

(71) Applicant: Abbott Diabetes Care, Inc., Alameda, CA (US)

(72) Inventors: Zenghe Liu, Alameda, CA (US); Tram Nguyen, Alameda, CA (US); Benjamin Feldman, Alameda, CA (US)

(21) Appl. No.: 18/621,449

(22) Filed: Mar. 29, 2024

Related U.S. Application Data
(60) Provisional application No. 63/493,595, filed on Mar. 31, 2023.
(60) Provisional application No. 63/493,600, filed on Mar. 31, 2023.

(51) Int. Cl. A61B 5/145; G01N 27/327

(52) U.S. Cl. CPC A61B 5/14546; G01N 27/3271; G01N 27/3277

(57) ABSTRACT

The present invention relates to analyte sensors for use in detecting the concentration of potassium ions in a biological fluid and methods of using the sensors.

The present application claims priority to United States Provisional Patent Application Ser. No. 63/493,595, filed Mar. 31, 2023, and 63/493,600, filed Mar. 31, 2023, the disclosures of which are herein incorporated by reference in their entireties.

### FIELD OF THE INVENTION

The present invention relates to analyte sensors for use in detecting the concentration of potassium ions in a biological fluid and methods of using the sensors.

### BACKGROUND OF THE INVENTION

The detection of various analytes within an individual can sometimes be vital for monitoring the condition of their health as deviations from normal analyte levels can be indicative of a physiological condition. For example, monitoring glucose levels allows people suffering from diabetes to take appropriate corrective action including administration of medicine or consumption of particular food or beverage products to avoid significant physiological harm. Other analytes such as potassium can be desirable to monitor for certain physiological conditions. In certain instances, it can be desirable to monitor more than one analyte to monitor single or multiple physiological conditions, particularly if a person is suffering from comorbid conditions that result in simultaneous dysregulation of two or more analytes in combination with one another.

Analyte monitoring in an individual can take place periodically or continuously over a period of time. Periodic analyte monitoring can take place by withdrawing a sample of bodily fluid, such as blood or urine, at set time intervals and analyzing ex vivo. Periodic, ex vivo analyte monitoring can be sufficient to determine the physiological condition of many individuals. However, ex vivo analyte monitoring can be inconvenient or painful in some instances. Moreover, there is no way to recover lost data if an analyte measurement is not obtained at an appropriate time. Continuous analyte monitoring can be conducted using one or more sensors that remain at least partially implanted within a tissue of an individual, such as dermally, subcutaneously, or intravenously, so that analyses can be conducted in vivo. Implanted sensors can collect analyte data on-demand, at a set schedule, or continuously, depending on an individual's particular health needs and/or previously measured analyte levels. Analyte monitoring with an in vivo implanted sensor can be a more desirable approach for individuals having severe analyte dysregulation and/or rapidly fluctuating analyte levels, although it can also be beneficial for other individuals as well.

Many analytes represent intriguing targets for physiological analyses, provided that a suitable detection chemistry can be identified. To this end, enzyme-based amperometric sensors configured for assaying glucose continuously in vivo have been developed and refined over recent years to aid in monitoring the health of diabetic individuals. Other analytes commonly subject to concurrent dysregulation with glucose in diabetic individuals include, for example, potassium. It can also be desirable to monitor potassium independent of glucose dysregulation as well. For example, potassium levels can be important to monitor in people suffering various kidney or heart diseases.

Implanted analyte sensors configured for detecting potassium in vivo are not currently available. Experimental systems which have been used for the in vitro detection of nanomolar potassium concentrations in fluids using ion-transfer stripping voltammetry are not suitable for detection of potassium in vivo for a variety of reasons. For instance, certain systems cannot detect the higher potassium concentrations found in biological fluids and also do not have sufficient sensor lifetime to be used in in vivo applications. Said systems are also too bulky for in vivo implantation and use, and are more difficult to manufacture.

Accordingly, there is a need in the art for sensors for detecting potassium in vivo. Since implanted analyte sensors often remain within a tissue of an individual for an extended period of time, it can be highly desirable for such analyte sensors to be made from stable materials exhibiting a high degree of biocompatibility.

### SUMMARY OF THE INVENTION

The present invention provides a potassium sensor that can be used in vivo for continuous monitoring and detection of potassium concentration in biological fluids. The potassium sensor does not comprise enzymes and is based upon non-enzymatic sensor chemistry.

In some embodiments, the potassium sensor comprises a working electrode with a potassium responsive active area disposed on a portion of an outer surface of the electrode. In some embodiments, the working electrode comprises a transition metal ion in a salt or complex form. In some embodiments, the potassium responsive active area comprises a potassium selective transport layer that forms an outer surface of the potassium responsive active area. In some embodiments, the potassium selective transport layer comprises a hydrophobic polymer and a potassium ionophore. In some embodiments, the sensor finds use in in vivo monitoring of potassium concentration in biological fluids.

In some embodiments, where the abovementioned sensor is used with a wide variety of electrochemical detection techniques such as amperometry, chronoamperometry and voltammetry, and also to enhance sensor lifetime for in vivo use, it is beneficial for the sensor to further comprise an electrolyte gel layer in between the outer surface of the working electrode and potassium selective transport layer. Without being limited by theory, it is believed that the electrolyte gel layer is improves sensor longevity. The electrolyte gel layer is also believed to be important for providing a favourable medium to facilitate reduction and oxidation reactions at the electrode surface that occur on operation of the potassium sensor.

Accordingly, in some embodiments, provided herein are systems, devices, and methods employs a sensor comprising: a first working electrode and a potassium responsive active area disposed on at least a portion of an outer surface of the first working electrode; wherein the portion of the outer surface of the first working electrode comprises a transition metal ion in a salt or complex form, and optionally a transition metal; and wherein the potassium responsive active area comprises: (i) a potassium selective transport layer forming an outer surface of the potassium responsive active area, wherein the potassium selective transport layer comprises a polymer, a potassium ionophore, a plasticizer, and an electrolyte; and (ii) an electrolyte gel layer disposed between the potassium selective transport layer and the portion of the outer surface of the first working electrode.

However, in some embodiments, a sensor as described above, but with no electrolyte gel layer, is provided and used in in vivo detection methods with no significant accompanying sacrifice of sensor lifetime or functionality on account of the omission of the electrolyte gel layer. Including an electrolyte gel layer in the sensor adds manufacturing cost and complexity when manufacturing the sensors. The ability to omit an electrolyte gel layer from the sensor provides greater ease of manufacture and cost savings and thus may be desirable in some instances.

The present invention is based, in part, on the surprising finding that when using certain specific electrochemical detection techniques, it is not necessary for the electrolyte gel layer to be included in in vivo potassium sensors of the type described above. Specifically, it has surprisingly been found that when the technique of chronoamperometry with short duration potentials is used with sensors of the type described above, the electrolyte gel layer may be omitted from the sensor without the functionality of the sensor being significantly compromised. This is highly surprising since with other electrochemical detection techniques, or when using chronoamperometry with longer duration potentials, the presence of the electrolyte gel layer has been found to be essential in providing good sensor functionality and lifetime for effective use in in vivo potassium concentration detection and monitoring in biological fluids.

Accordingly, in some embodiments, the present invention provides systems, devices, and methods for detecting potassium ions in a fluid comprising: (a) providing an analyte sensor comprising: a first working electrode and a potassium responsive active area disposed on at least a portion of an outer surface of the first working electrode; wherein the portion of the outer surface of the first working electrode comprises a transition metal ion in a salt or complex form, and optionally a transition metal; and wherein the potassium responsive active area comprises: a potassium selective transport layer forming an outer surface of the potassium responsive active area, wherein the potassium selective transport layer comprises a polymer, a potassium ionophore, a plasticizer, and an electrolyte; and (b) applying a potential to the first working electrode; (c) obtaining a signal indicative of oxidation and/or reduction of the transition metal ion in a salt or complex form, and optionally transition metal of the first working electrode; wherein the signal is indicative of the concentration of potassium ions in the fluid; and (d) determining the concentration of potassium ions in the fluid from the signal obtained in step (c); wherein steps (b) and (c) comprise using chronoamperometry to obtain the signal indicative of the concentration of potassium ions in the fluid, wherein the chronoamperometry comprises the successive application of first and second potentials; wherein the first potential is applied for a duration of from 10 milliseconds to 60 seconds, and wherein the second potential is applied for a duration of from 10 milliseconds to 60 seconds; and wherein the first potential is different from the second potential.

It has been found that when chronoamperometry comprising application of potentials of from 10 milliseconds to 60 seconds in duration is used, there is no need to include an electrolyte gel layer disposed between the potassium selective transport layer (or electrolyte gel layer if included). Successive application of a positive potential to the electrode then causes oxidation (i.e., the reverse of the process that occurs on application of negative potential) which causes the counterion (e.g., a chloride anion) to migrate back on to the electrode surface from the potassium selective transport layer (or electrolyte gel layer if included) to regenerate the original transition metal salt. Without being limited by theory, it is believed that where longer durations of negative potential are applied, the counterions (e.g., chloride ion) migrate from the electrode surface into the potassium selective transport layer or electrolyte gel layer and also passively diffuse away from the electrode surface. The longer the negative potential is applied for, the further the counterions diffuse away from the electrode surface. This means that when a subsequent positive potential is applied, it is harder to recover the counterions on the electrode surface. Over time, this leads to incomplete recovery of the counterions on the electrode and incomplete regeneration of the transition metal salt during the positive potentials. This incomplete generation may compromise sensor functionality overtime. Surprisingly, it has been found that using longer positive potentials than negative potentials alleviates this problem leading to improved sensor lifetime.

In some embodiments, both the negative potentials and positive potentials applied comprise potentials of from 10 milliseconds to 5 seconds in duration and the positive and negative potentials applied are of different duration. In some embodiments, the positive potentials are applied for a longer duration than the negative potentials.

In some embodiments, the positive potentials are applied for a longer duration than the negative potentials and the positive potentials are applied for a duration of from 2 seconds to 5 seconds, and the negative potentials are applied for a duration of from 1 second to 2 seconds.

In some embodiments, the chronoamperometry comprises the successive application of alternating positive and negative potentials, wherein the potentials are applied for a duration of from 10 milliseconds to 60 seconds. In some embodiments, the chronoamperometry comprises the successive application of alternating positive and negative potentials, wherein the potentials are applied for a duration of from 500 milliseconds to 60 seconds. In some embodiments, the chronoamperometry comprises the successive application of alternating positive and negative potentials, wherein the potentials are applied for a duration of from 500 milliseconds to 20 seconds.

In some embodiments, the duration of the positive potentials is different to the duration of the negative potentials. In some embodiments, the positive potentials are applied for a longer duration than the negative potentials.

In some embodiments, the duration of the positive potentials is from 2 seconds to 5 seconds, and wherein the duration of the negative potentials is from 1 second to 2 seconds.

In some embodiments, the chronoamperometry comprises the successive application of alternating positive and negative potentials; wherein the successive negative potentials or positive potentials comprise potentials of from 50 mV to 500 mV; for example, from 100 mV to 300 mV; for example, from 150 mV to 250 mV.

In some embodiments, the chronoamperometry comprises the successive application of alternating positive and negative potentials; wherein the negative potentials or positive potentials comprise potentials of from 175 mV to 225 mV; for example, 200 mV.

In some embodiments, the positive potentials are applied for a duration of 3 seconds and the negative potentials are applied for a duration of 1 second. In some embodiments, for example, the negative potential is 200 mV and the positive potential is also 200 mV.

The advantages described above associated with the duration of the positive potentials being longer than the duration of the negative potentials are also observed where the first and second potential are both positive potentials or where the first and second potentials are both negative potentials. Accordingly, in an embodiment, the first potential is applied for a different duration to the second potential. In some embodiments, the potential associated with reduction at the electrode is applied for a shorter duration than the potential associated with oxidation at the electrode.

In some embodiments, the chronoamperometry comprises pulse amperometry.

The term pulse amperometry as used herein is used to refer to any electrochemical detection technique known in the art as being encompassed by the term pulse amperometry or pulsed amperometry. In pulse amperometry, chronoamperometry using very short potential steps is carried out (e.g., potential steps with a duration in accordance with the method of the invention). Each potential step generates a resultant current signal that can be measured over time. For each potential step, it is the current signal generated during the latter part of the duration of each potential step that is indicative of potassium concentration of the fluid in which the sensor is disposed. For each potential, pulse amperometry essentially samples the current generated during this latter part of the duration of each potential step. Pulse amperometry may include determining an average current value of the current generated over time for the relevant latter part of the duration of the potentials. Pulse amperometry thus provides a single current value (e.g., an average current) for each potential. An advantage of using pulse amperometry in this way is that it makes it easier to compare the current produced for potential steps at different potassium concentrations.

For any of the above configurations, in some embodiments, the fluid comprising the potassium ions that are detected comprises a biological fluid. In some embodiments, the biological fluid comprises dermal fluid, interstitial fluid, plasma, blood, lymph, synovial fluid, cerebrospinal fluid, saliva, bronchoalveolar lavage or amniotic fluid.

In some embodiments, the sensor, or at least a portion of the sensor (outer surface of the first working electrode), is located in vivo. In some such embodiments, the methods comprise determining the concentration of potassium ions in an in vivo biological fluid.

The terms outer and inner as used herein in the context of respective surfaces and layers are used in relation to the first working electrode. The term inner as used herein is used to mean nearer to the first working electrode. Accordingly, when present, the electrolyte gel layer of the potassium responsive active area, in some embodiments, may be described as being disposed inner to the potassium selective transport layer of the potassium responsive active area because it is between the potassium selective transport layer and the working electrode. The term outer as used herein is used to refer to being further away from the first working electrode and nearer to an analyte containing fluid in which the sensor is disposed.

In some embodiments, the potassium responsive active area comprises only the potassium selective transport layer, with no additional layers present. Accordingly, in some embodiments, the potassium responsive active area does not comprise an electrolyte gel layer. However, in other embodiments, the potassium responsive active area may comprise one or more additional layers, such as one or more layers inner, outer, or both inner and outer to the potassium selective transport layer, provided of course that the presence of any additional layers does not impede the functionality of the sensor.

As discussed in further detail below, in some embodiments, the potassium responsive active area may further comprise an electrolyte gel layer. The potassium responsive active area thus in some embodiments comprises an outer potassium selective transport layer and an inner electrode gel layer between the potassium selective transport layer and the working electrode. In some embodiments, no additional layers are between these layers and the potassium selective transport layer is directly adjacent to the electrolyte gel layer and the electrolyte gel layer is directly adjacent to the first working electrode. However, this is not essential, and the sensor may comprise additional layers between the first working electrode and electrolyte gel layer, and/or additional layers between the electrolyte gel layer and the potassium selective transport layer, provided of course that the presence of any additional layers do not impede the functionality of the sensor.

The polymer of the potassium selective transport layer can be any suitable polymer. In some embodiments, the polymer is a polymer that is resistant to biological fluids in which the sensor is disposed. Suitable polymers will be apparent to the skilled person and include polyvinylchloride (PVC), polyethylene, polypropylene, polybutylene, polyurethane, and other suitable hydrophobic polymers. In some embodiments, the polymer comprises polyvinylchloride (PVC). In some embodiments, the polymers have a weight average molecular weight of from 50,000 Da to 500,000 Daltons. In some embodiments, the polymer comprises polyvinyl chloride with a weight average molecular weight of from 50,000 Daltons to 500,000 Daltons. In some embodiments, polyvinylchloride with a weight average molecular weight of 200,000 Da is used.

The potassium selective transport layer further comprises a plasticizer. In some embodiments, the plasticizer is a hydrophobic plasticizer. In some embodiments, the plasticizer comprises a phthalate compound, an adipate compound, a glutarate compound, a sebacate compound, a phosphate compound, a trimellitate compound, an epoxy compound, or a combination thereof. In some embodiments, the plasticizer comprises 2-nitrophenyloctylether (NPOE). In some embodiments, the polymer comprises polyvinylchloride and the plasticizer comprises a PVC plasticizer such as those discussed above. In some embodiments, the polymer comprises polyvinylchloride and the plasticizer comprises 2-nitrophenyloctylether (NPOE). Specific examples of other plasticizers that can be used include dinonyladipinate (DNA), tris(2-ethylhexyl) phosphate (TEHP), tris(ethylhexyl) phosphate (TEHP), bis (2-ethylhexyl) adipate (DOA), dioctylphthalate (DOP), and bis (2-ethylhexyl) sebacate (DOS). The plasticizers should be compatible with the polymer and components of the electrodes. They should also be soluble in solvents used for the preparation of the potassium selective transport layer. The plasticizer functions so as to soften the polymer and make it accessible to potassium ions that are present in a biological fluid in which the sensor is disposed.

The polymer and plasticizer are present in any suitable ratio. Suitable ratios will be appreciated by those skilled in the art. In some embodiments, the polymer and plasticizer of the potassium selective transport layer are present in a weight ratio of from 1:1 to 1:10; for example, from 1:2 to 1:8; for example, from 1:3 to 1:5.

The potassium selective transport layer also comprises a potassium ionophore. Any suitable potassium ionophore may be used. Suitable potassium ionophores are those that have a greater binding affinity and selectivity for potassium ions over other cations commonly found in biological fluids such as sodium.

In some embodiments, the potassium ionophore comprises valinomycin, Gramicidin A, laidlomycin, lasalocid, maduramicin, monensin, Narasin, Nigericin, Nonactin, Nystatin, Salinomycin, a crown ether, a cryptand, or any combination thereof. In some embodiments, the potassium ionophore is valinomycin, Gramicidin A, laidlomycin, lasalocid, maduramicin, monensin, Narasin, Nigericin, Nonactin, Nystatin, Salinomycin, a crown ether, a cryptand, or any combination thereof. In some embodiments, the potassium ionophore may comprise or may be a molecule that comprises an ionophoric moiety, such as a conjugate or derivative of the specific molecules and types of molecules listed above. For example, the ionophore may be or may comprise a valinomycin moiety conjugated to one or more separate non-ionophoric moieties.

In some embodiments, the potassium ionophore comprises a hydrophobic ionophore that is capable of being dispersed within the polymer of the potassium selective transport layer. In some embodiments, the potassium ionophore comprises valinomycin, or is valinomycin.

The term potassium ionophore as used herein is used to refer to a chemical species that reversibly binds potassium ions. In some embodiments, ionophores have a cyclic molecular structure and the potassium ion is reversibly bound to the ionophore by being located in the centre of the cyclic structure.

The potassium ionophore is present in the potassium selective transport layer in any suitable concentration. In some embodiments, the potassium ionophore is present in the potassium selective transport layer in an amount of from 0.1% to 30% by weight of the polymer present in the potassium selective transport layer; for example, from 2% to 20% by weight; for example, from 5% to 15% by weight.

The potassium selective transport layer further comprises an electrolyte. In some embodiments, the electrolyte comprises a hydrophobic electrolyte. In some embodiments, the electrolyte is insoluble in water. In some embodiments, the electrolyte comprises a salt comprising one or more organic cations and one or more organic anions. In some embodiments, the one or more organic cations and one or more organic anions are sterically bulky. In some embodiments, electrolytes comprising sterically bulky cations and anions of an organic nature are hydrophobic, which is highly desirable. In some embodiments, the electrolyte is relatively hydrophobic so that it stays within the potassium selective transport layer and does not leach out into biological fluids within which the sensor is disposed, as discussed in further detail below.

In some embodiments, the electrolyte comprises a salt comprising a quaternary ammonium cation with the formula R₁R₂R₃R₄N⁺, wherein R₁ to R₄ are each independently selected from C₁ to C₃₀ straight chain or branched alkyl or alkenyl groups, optionally substituted by one to three groups selected from: C₁ to C₄ alkoxy, C₂ to C₈ alkoxyalkoxy, C₃ to C₆ cycloalkyl, —OH, —NH₂, —SH, —CO₂(C₁ to C₆)alkyl, and —OC(O)(C₁ to C₆)alkyl; for example, wherein R₁ to R₄ are each independently selected from C₅ to C₁₅ alkyl groups, optionally substituted as described above. In some embodiments, R₁ to R₄ are each independently selected from C₄ to C₁₂ straight chain or branched alkyl groups, optionally substituted as described above. In some embodiments, the electrolyte comprises a salt comprising a tetraoctylammonium cation.

In some embodiments, the electrolyte comprises a salt comprising an anion of the formula BX₄⁻ or PX₆, wherein each X is independently selected from C₅ to C₁₅ aliphatic or aromatic hydrocarbyl groups, optionally substituted by one to three groups selected from: C₁ to C₄ alkoxy, C₂ to C₈ alkoxyalkoxy, C₃ to C₆ cycloalkyl, —OH, —NH₂, —SH, —CO₂(C₁ to C₆)alkyl, F, Cl and —OC(O)(C₁ to C₆)alkyl. In some embodiments, each X is independently selected from C₅ to C₁₅ aromatic groups, C₅ to C₁₅ alkyl groups, or C₅ to C₁₅ alkenyl groups; optionally substituted as described above. In some embodiments, each X is independently selected from C₅ to C₉ aromatic groups; optionally substituted as described above. In some embodiments, each X is independently selected from C₆ aromatic groups; optionally substituted as described above. In some embodiments, the electrolyte comprises a tetrakis(pentafluorophenyl)borate anion.

In some embodiments, the electrolyte comprises tetraoctylammonium tetrakis(pentafluorophenyl)borate (TOATB).

The electrolyte is present in the potassium selective transport layer in any suitable concentration. In some embodiments, the electrolyte is present in the potassium selective transport layer in a concentration of from 5 mM to 50 mM; for example, from 5 mM to 20 mM; for example, from 5 mM to 15 mM.

As discussed above, in some embodiments, the sensor and/or the potassium responsive active area do not comprise an electrolyte gel layer. However, in some embodiments, the potassium responsive active area may comprise an electrolyte gel layer.

In some embodiments, the potassium selective transport layer comprises PVC, valinomycin, tetraoctylammonium tetrakis(pentafluorophenyl)borate (TOATB) and 2-nitrophenyloctylether (NPOE).

When included, in some embodiments, the electrolyte gel layer comprises an electrolyte hydrogel layer. In some embodiments, the electrolyte gel layer comprises an electrolyte hydrogel layer comprising an aqueous electrolyte solution and a crosslinked hydrophilic polymer. Any suitable hydrophilic polymer can be used that forms a hydrogel when crosslinked. In some embodiments, the crosslinked hydrophilic polymer comprises crosslinked polyvinyl alcohol, crosslinked polyethylene glycol, a crosslinked acrylate polymer, a crosslinked acrylamide polymer, a crosslinked hyaluronic polymer, a crosslinked chitosan, a crosslinked heparin, a crosslinked alginate, or a crosslinked fibrin. In some embodiments, the crosslinked hydrophilic polymer comprises a crosslinked acrylamide polymer. In some embodiments, the crosslinked hydrophilic polymer comprises a crosslinked N, N-dimethylacrylamide polymer.

In some embodiments, the crosslinked hydrophilic polymer is formed by reaction of a hydrophilic monomer and a crosslinking agent. Any suitable ratio of crosslinking agent to hydrophilic monomer may be used. In some embodiments, the hydrophilic monomer and crosslinking agent are reacted in an amount of from 90 mol % to 99 mol % hydrophilic monomer and from 1 mol % to 10 mol % crosslinking agent; for example, from 95 mol % to 99 mol % hydrophilic monomer and from 1 mol % to 5 mol % crosslinking agent. The hydrophilic monomers and crosslinking agents react to form a crosslinked hydrophilic polymer. In this regard, the monomers react with each other in a polymerization reaction to form a polymer chain and the crosslinking agent crosslinks the polymer chains.

Suitable crosslinking agents will be apparent to those of skill in the art. In some embodiments, the crosslinking agent comprises ethylene glycol dimethacrylate. Accordingly, in some embodiments, the crosslinked hydrophilic polymer comprises N, N-dimethylacrylamide crosslinked by ethylene glycol dimethacrylate.

The aqueous electrolyte of the electrolyte gel layer may be any suitable electrolyte or combination of suitable electrolytes. In some embodiments, the aqueous electrolyte comprises a solution of an aqueous solution of a Group 1 metal salt; for example, an aqueous solution of a Group 1 metal halide salt; for example, an aqueous solution of lithium chloride. Alternatively, the aqueous electrolyte solution comprises a quaternary ammonium halide salt. In some embodiments, the aqueous electrolyte solution comprises (i) an aqueous solution of a Group 1 metal salt; for example, an aqueous solution of a Group 1 metal halide salt; for example, an aqueous solution of lithium chloride; and (ii) a quaternary ammonium halide salt. In some embodiments, the quaternary ammonium halide salt comprises a tetra C₄ to C₁₂ alkyl ammonium halide salt such as tetraoctylammonium chloride.

In some embodiments, the aqueous electrolyte solution comprises an aqueous solution of a Group 1 metal salt as discussed above, wherein the Group 1 metal salt is present in the solution at a concentration of from 50 mM to 150 mM; for example, from 75 mM to 125 mM.

In some embodiments, where the aqueous electrolyte solution comprises both a Group 1 metal salt as described above and also a quaternary ammonium halide salt as described above, the quaternary ammonium halide salt is present in the aqueous electrolyte solution at a concentration of from 1 mM to 5 mM.

The aqueous electrolyte solution may also comprise a buffering agent. Any suitable buffering agent may be used. An example of a buffering agent that may be used is (4-(2-hydroxyethyl)-1-piperazineethanesulfonic acid) (HEPES). The buffering agent may be present at any suitable concentration. In some embodiments, if present, the buffering agent is present in the electrolyte solution at a concentration of from 1 mM to 5 mM.

The aqueous electrolyte solution and crosslinked hydrophilic polymer are present in the electrolyte gel layer in any suitable amount to form a hydrogel layer. In some embodiments, the aqueous electrolyte solution is present in the electrolyte hydrogel layer in an amount of from 60% to 95% by weight; and the crosslinked hydrophilic polymer is present in the electrolyte hydrogel layer in an amount of from 5% to 40% by weight. In some embodiments, the aqueous electrolyte solution is present in the electrolyte hydrogel layer in an amount of from 70% to 90% by weight; and the crosslinked hydrophilic polymer is present in the electrolyte hydrogel layer in an amount of from 10% to 30% by weight.

The first working electrode comprises a portion on its outer surface, wherein the portion comprises a transition metal ion in a salt or complex form, and optionally a transition metal.

In some embodiments, the transition metal ion in a salt or complex form, and optionally a transition metal comprises silver, gold, osmium, iron, cobalt, nickel, copper, ruthenium, platinum, an ion thereof, or any combination thereof. In some embodiments, the transition metal ion in a salt or complex form, and optionally a transition metal comprises silver, gold, osmium, an ion thereof, or a combination thereof. In some embodiments, the transition metal ion in a salt or complex form, and optionally a transition metal comprises silver or osmium, or an ion thereof.

Where the portion of the outer surface of the first working electrode comprises a transition metal ion in its salt or complex form and a transition metal, in some embodiments, both the transition metal and the transition metal ion are the same element. For example, the outer surface portion can comprise silver metal and a silver ion in salt or complex form. In some embodiments, in these embodiments, the outer surface portion comprises silver metal and a silver salt such as a silver halide salt. In some embodiments, the outer surface portion comprises silver metal and silver chloride.

Where the portion of the outer surface of the first working electrode comprises a transition metal ion in its salt or complex form but does not comprise a transition metal, in some embodiments, the electrode portion comprises a first transition metal ion in a first oxidation state and a second transition metal ion of the same element in a second oxidation state. For example, the electrode portion can comprise the osmium ions Os (II) and Os (III). In such embodiments, both the first and second transition metal ions are in salt or complex form.

The transition metal ion of the portion of the outer surface of the first working electrode may be in salt form. The salts can comprise any suitable salt. In some embodiments, the salts comprise halide salts, for example, chloride salts. For example, the salts may comprise silver chloride, or chloride salts of any of the other metals listed above.

The transition metal ion of the portion of the outer surface of the first working electrode may be in complex form. The term complex form as used herein is used to refer to the transition metal ion being coordinated to one or more ligands. The resultant complex may have a positive, negative, or neutral electrostatic charge depending on the sum of the charges of the transition metal ion and ligands associated therewith. In some embodiments, the complex has a positive electrostatic charge. Where the complex has a positive or negative electrostatic charge, the complex will also have one or more counterions associated therewith. In this respect, the complex will be a salt comprising a complex ion and a counterion. In some embodiments, where the complex ion has a positive charge, the complex will be in salt form and associated with one or more anionic counterions. Examples of suitable anionic counter ions include halide ions and other suitable anions. In some embodiments, the counterions comprise chloride ions.

Examples of suitable complexes of transition metal ions include those of the formula:

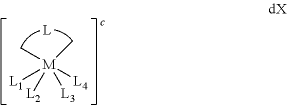

wherein M is one of the metals discussed above, for example, osmium;
L is selected from the group consisting of:

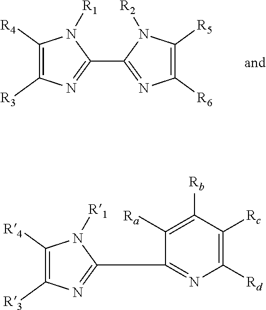

R₁, R₂, and R′₁ are independently substituted or unsubstituted alkyl, alkenyl, or aryl groups, for example, wherein R₁, R₂, and R′₁ are unsubstituted C₁ to C₁₂ alkyl groups; R₃, R₄, R₅, R₆, R′₃, R′₄, Rₐ R_{b}, R_{c}, and R_{d} are independently selected H, F, Cl, Br, I, NO₂, CN, CO₂H, SO₃H, NHNH₂, SH, OH, NH₂, or substituted or unsubstituted alkoxycarbonyl, alkylaminocarbonyl, dialkylaminocarbonyl, alkoxy, alkylamino, dialkylamino, alkanoylamino, arylcarboxamido, hydrazino, alkylhydrazino, hydroxylamino, alkoxylamino, alkylthio, alkenyl, aryl, or alkyl; c is an integer selected from −1 to −5 or +1 to +5 indicating a positive or negative charge;
X represents at least one counterion;
d is an integer from 1 to 5 representing the number of counter ions, X;
and L₁, L₂, L₃, and L₄ are ligands;
wherein L₁ comprises a heterocyclic compound coupled to a polymeric backbone, and wherein L₁ and L₂ in combination form a first bidentate ligand.

In some embodiments, L₃ and L₄ in combination form a second bidentate ligand.

In some embodiments, at least one of the first bidentate ligand or second bidentate ligand is selected from the group consisting of substituted or unsubstituted 2, 2′-bipyridines, 2,2′-biimidazoles, and 2-(2-pyridyl) imidazoles.

In some embodiments, the second bidentate ligand is selected from the group consisting of substituted or unsubstituted 2, 2′-bipyridines, 2,2′-biimidazoles, and 2-(2-pyridyl) imidazoles.

In some embodiments, the first bidentate ligand is selected from the group consisting of substituted 2, 2′-bipyridines, 2,2′-biimidazoles, and 2-(2-pyridyl) imidazoles. In some embodiments, the substituted 2, 2′-bipyridines, 2,2′-biimidazoles, or 2-(2-pyridyl) imidazoles are coupled to the polymeric backbone via substituents. In some embodiments, these substituents comprise substituted C1 to C12 alkyl groups; substituted C1 to C12 alkoxy groups; C1 to C12 alkylthio groups; C1 to C12 alkylamino groups; C2 to C24 dialkylamino groups; or C1 to C12 alkyl groups.

In some embodiments, the polymeric backbone is selected from the group consisting of styrene/maleic anhydride copolymer, methylvinylether/maleic anhydride copolymer, poly(4-vinylbenzylchloride) copolymer, poly(allylamine) copolymer, poly(4-vinylpyridine) copolymer, poly (4-vinylpyridine), poly (N-vinylimidazole), and poly (4-styrene sulfonate). Further details of such complexes are disclosed in U.S. Pat. No. 6,605,201, herein incorporated by reference in its entirety.

The complexes, such as but not limited to those discussed above, may be in the form of a redox polymer comprising a polymer backbone grafted to a complex such as but not limited to those discussed above. In some embodiments, the redox polymer comprises a polymeric backbone coupled to a transition metal ion complex as described above. In some embodiments, the redox polymer comprises a polymeric backbone coupled to a transition metal ion complex as described above where L₁ is a ligand comprising a heterocyclic compound coupled to the polymeric backbone. In some embodiments, the redox polymers are crosslinked and comprise a cross-linker. Further details of such redox polymers are disclosed in US2012/0132525, herein incorporated by reference in its entirety.

In some embodiments, the polymeric backbone is a poly(vinylpyridine). For example, a poly(vinylpyridine) having the following general structure may be used.

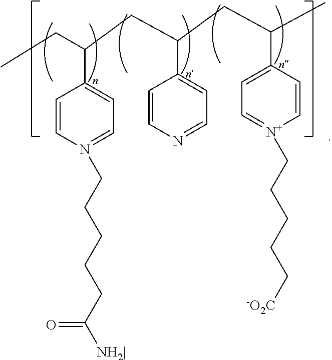

In some embodiments, in the structure depicted above, n is 2, n′ is 17 and n″ is 1. In some embodiments, the polymeric backbone is crosslinked. For example, the polymeric backbone may be crosslinked by a poly(ethylene glycol) diglycidyl ether (PEGDGE), such as a PEGDGE of the following formula where n is an integer of from 1 to 500.

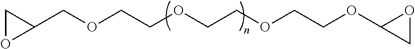

The purpose of including the transition metal ion in a complex or redox polymer grafted to a complex such as those discussed above is that, firstly, the oxidation/reduction potential of the ions are altered such that oxidation/reduction by application of potential from the electrode is made easier. The presence of the complex/redox polymer thus facilitates oxidation and/or reduction of the ions. Additionally, the complexes and redox polymers are useful in that they serve to hold the ions in place on the surface of the working electrode so that they are more easily reduced and/or oxidised by application of potential from the electrode.

It is not essential that the transition metal ion is in complex form. For example, as discussed above, in some embodiments, the portion of the outer surface of the working electrode comprises silver, for example, silver ions in the form of silver salts such as halides (e.g., chloride) (i.e., the silver ions are not in complex form).

Where the transition metal ion in a salt or complex form, and optionally a transition metal comprises osmium ions, in some embodiments, the osmium ions are in the form of a complex or redox polymer such as those discussed above.

For example, in some embodiments, the electrode portion comprises the ions osmium (II) and/or osmium (III), for example, osmium (II) and osmium (III). In some embodiments, the osmium ions are in complex form. In some embodiments, the osmium ions are in the form of a redox polymer coupled to a ligand that is complexed with the osmium (II) and/or osmium (III) ion. In some embodiments, the redox polymer is as described above. In some embodiments, the counterions to the redox polymer are chloride anions.

The sensor may further a support layer on which the first working electrode is disposed; for example, wherein the support layer comprises a polymer such as PET.

Where the portion of the outer surface of the first working electrode comprises silver and silver chloride (AgCl) as described above, in some embodiments, the first working electrode comprises a silver/silver chloride electrode layer. In some embodiments, the silver/silver chloride is located along a length of the first working electrode (e.g., along the length of the silver/silver chloride electrode layer). In such embodiments, where the potassium responsive active area is disposed on the portion of the outer surface of the first working electrode, but not along the entire length of the outer surface of the first working electrode, the silver/silver chloride may be located both in the portion on which the potassium selective transport layer is disposed and also in a portion of the outer surface of the first working electrode on which the potassium responsive active area is not disposed.

Where the first working electrode comprises a silver/silver chloride electrode layer, the sensor may further comprise a support layer on which the silver/silver chloride electrode layer is disposed. In some embodiments, the support layer comprises a polymer such as PET.

In embodiments where the portion of the first working electrode comprises a complex of osmium (II), a complex of osmium (III), or a combination thereof, the first working electrode may comprise a carbon electrode layer and the portion of the outer surface of the first working electrode comprises a layer of the osmium ion complex/complexes, disposed upon a portion of an outer surface of the carbon electrode layer. In some embodiments, the sensor further comprises a support layer on which the carbon electrode layer is disposed. In some embodiments, the support layer comprises a polymer such as PET.

The layers of the potassium responsive active area can have any suitable thickness.

In some embodiments, the potassium selective transport layer has a thickness of from 1 to 200 μm.

In some embodiments, the electrolyte gel layer, if included, has a thickness of from 1 to 50 μm.

In some embodiments, the first working electrode has a thickness of from 1 nm to 20 μm.

In some embodiments, the sensor further comprises a layer of dielectric material disposed on a second portion of the outer surface of the first working electrode to define an exposed area of the working electrode where the potassium responsive active area is disposed on. Any suitable dielectric layer known to be suitable for use with analyte sensors for in vivo use may be used. Examples of dielectric materials that can be used include the material Dupont 5018. In some embodiments, the potassium selective transport layer forms the outer layer of the sensor and is in direct contact with biological fluid in which the sensor is disposed. In these embodiments, the dielectric material, if present, will also form part of the outer surface of the sensor.

In other embodiments, the sensor further comprises a mass transport limiting membrane. For example, the first working electrode and potassium responsive active area may be fully encapsulated or partially encapsulated by a mass transport limiting membrane. In some embodiments, the mass transport limiting membrane comprises a polymer membrane. The polymer membrane may comprise any suitable polymer. In some embodiments, the polymer membrane comprises polyvinylpyridine, polyvinylimidazole, a copolymer of vinylpyridine and styrene, or a combination thereof. In some embodiments, the polymer of the polymer membrane is crosslinked. Any suitable crosslinking agent may be used. In some embodiments, the polymer of the polymer membrane is crosslinked by reaction with a polyethylene glycol tetraglycidyl ether. The mass transport limiting membrane is of course permeable to potassium so that the potassium from a fluid in which the sensor is disposed can access the potassium responsive active area.

The sensor may further comprise one or more additional active areas. The additional active areas may be as described above. Alternatively, the additional active areas may comprise different chemistries and be tailored for the detection and concentration measurement of other analytes. Any suitable detection chemistries for the detection of other analytes known in the art may be used.

The sensor may further comprise one or more additional working electrodes and/or one or more additional active areas. Optionally, the one or more additional working electrodes or one or more additional active areas are as described above. Alternatively, the one or more additional working electrodes or one or more additional active areas may comprise different chemistries and be tailored for the detection and concentration measurement of other analytes. Any suitable detection chemistries for the detection of other analytes known in the art may be used.

Accordingly, the one or more additional active areas may be configured to be responsive to potassium; or configured to be responsive to one or more additional analytes.

In some embodiments, the sensor is an in vivo analyte sensor, wherein the sensor or at least a portion of the sensor is suitable for in vivo implantation. In some embodiments, the sensor comprises a sensor tail that comprises the first working electrode and any other working electrodes if present.

In some embodiments, the sensor is suitable for detecting the concentration of potassium in a biological fluid; for example, wherein the biological fluid comprises dermal fluid, interstitial fluid, plasma, blood, lymph, synovial fluid, cerebrospinal fluid, saliva, bronchoalveolar lavage or amniotic fluid.

The analyte sensor may have any suitable design and set up for the in vivo detection of potassium and/or determination of potassium concentration, as described in further detail below. Further details of analyte sensor designs will be apparent to those skilled in the art from the benefit of this disclosure.

In some embodiments, in addition to a sensor tail, the sensor further comprises a sensor housing; an adhesive layer in contact with the sensor housing adapted for adhering the sensor housing to a tissue surface; and wherein the sensor tail protrudes from the sensor housing through the adhesive layer. However, it will be appreciated that alternative designs and variations of this design may also be used provided that the sensor is still suitable for detection of in vivo potassium.

The analyte sensor may further comprise a counter electrode, a reference electrode, or both.

In some embodiments, the sensor has a length of from 5 mm to 10 mm; a width of from 0.23 mm to 0.5 mm (e.g., 0.3 mm to 0.5 mm); and a thickness of from 0.1 mm to 0.4 mm (e.g., 0.2 mm to 0.4 mm).

In some embodiments, the signal obtained is indicative of oxidation of a transition metal atom or ion to a higher oxidation state; or indicative of reduction of a transition metal ion to a lower oxidation state. For example, the signal may be indicative of oxidation of a transition metal atom with an oxidation state of 0 to an oxidation state of +1; indicative of oxidation of a transition metal atom with an oxidation state of +1 to an oxidation state of +2; or indicative of oxidation of a transition metal atom with an oxidation state of +2 to an oxidation state of +3. Alternatively or additionally, the signal may be indicative of reduction of a transition metal ion with an oxidation state of +1 to 0; indicative of reduction of a transition metal ion with an oxidation state of +2 to +1; or indicative of reduction of a transition metal ion with an oxidation state of +3 to +2.

In some embodiments, the signal may be indicative of oxidation of a silver atom with an oxidation state of zero to a silver ion with an oxidation state of +1; and/or indicative of reduction of a silver ion with an oxidation state of +1 to a silver atom with an oxidation state of zero. Alternatively, the signal may be indicative of oxidation of an osmium ion with an oxidation state of +2 to an osmium ion with an oxidation state of +3; and/or indicative of reduction of an osmium ion with an oxidation state of +3 to an osmium ion with an oxidation state of +2.

In some embodiments, the transition metal ion in a salt or complex form, and optionally a transition metal of the portion of the outer surface of the first working electrode comprises silver and/or silver chloride (AgCl), as described above; and wherein the signal is indicative of reduction of silver chloride (AgCl) to silver, and/or oxidation of silver to silver chloride (AgCl).

Alternatively, the transition metal ion in a salt or complex form, and optionally a transition metal of the outer surface of the first working electrode comprises a complex of osmium (II), a complex of osmium (III) or both, as described above; and wherein the signal is indicative of reduction of osmium (III) chloride to osmium (II) chloride, and/or oxidation of osmium (II) chloride to osmium (III) chloride.

Provided herein are uses of any of the sensors described above or herein, for example, for the detection of potassium ion concentration in a fluid. In some embodiments, the fluid is a biological fluid. In some embodiments, the biological fluid and the analyte sensor are in vivo. In some embodiments, the biological fluid comprises dermal fluid, interstitial fluid, plasma, blood, lymph, synovial fluid, cerebrospinal fluid, saliva, bronchoalveolar lavage or amniotic fluid.

The method of detecting may comprise any suitable electrochemical detection method.

For example, in some embodiments, the method of detecting comprises a voltametric method comprising applying a varying potential to the first working electrode; wherein the detected signal comprises a peak current measurement. Alternatively or additionally, the method of detecting comprises an amperometric method; comprising applying a constant potential to the first working electrode and measuring the resultant current. Alternatively or additionally, the method of detecting comprises a chronoamperometric method; comprising applying a potential that alternates between two different values and measuring the resultant current.

### DESCRIPTION OF THE DRAWINGS

FIG. 1 shows a diagram of an illustrative sensing system that can incorporate an analyte sensor of the present disclosure.

FIGS. 2A-2C show cross-sectional diagrams of analyte sensors including a single active area.

FIGS. 3A-3C show cross-sectional diagrams of analyte sensors including two active areas.

FIG. 4 shows a cross-sectional diagram of an analyte sensor including two active areas.

FIGS. 5A-5C show perspective views of analyte sensors including two active areas upon separate working electrodes.

FIGS. 6 and 7 show example sensor chemistries of the present invention. FIGS. 6a and 7a show embodiments that employ a gel layer and FIGS. 6b and 7b show embodiments without a gel layer.

FIG. 8 compares the current observed at different voltages where voltammetry is used to determine potassium concentration in a fluid with a sensor of the present disclosure and a comparative sensor comprising no potassium ionophore.

FIGS. 9 and 10 show the difference in current and peak current when using voltammetry to determine the potassium concentration of a fluid in which a sensor of the present disclosure is disposed.

FIG. 11 shows the effect of voltage on current in a voltammetry experiment using a comparative sensor that does not comprise a potassium ionophore.

FIG. 12 shows current as a function of time in an amperometry method carried out using a sensor of the present disclosure.

FIGS. 13 and 14 show current as a function of time in chronoamperometry methods carried out using a sensor of the present disclosure.

FIGS. 15 and 16 show current as a function of time in chronoamperometry methods carried out using a sensor of the present disclosure.

FIGS. 17 and 18 show current as a function of time in chronoamperometric methods of the present invention using sensors of the present disclosure.

FIG. 19 shows the data of FIG. 18 sampled with pulse amperometry.

FIG. 20 shows sensors of the present disclosure tested using pulse amperometry methods at different potassium concentrations.

FIGS. 21 and 22 show current as a function of time in pulse amperometry methods of the present invention using sensors of the present disclosure, where the sensors are tested on successive days.

### DETAILED DESCRIPTION OF THE INVENTION

The present disclosure provides sensor chemistries suitable for monitoring potassium levels over a range of physiologically relevant potassium concentrations, particularly detection chemistries utilizing non-enzymatic systems.

### Definitions

The terms used in this specification generally have their ordinary meanings in the art, within the context of this disclosure and in the specific context where each term is used. Certain terms are discussed below, or elsewhere in the specification, to provide additional guidance to the practitioner in describing the compositions and methods of the present disclosure and how to make and use them.

As used herein, the use of the word “a” or “an” when used in conjunction with the term “comprising” in the claims and/or the specification can mean “one,” but it is also consistent with the meaning of “one or more,” “at least one,” and “one or more than one.” The terms “comprise(s),” “include(s),” “having,” “has,” “can,” “contain(s),” and variants thereof, as used herein, are intended to be open-ended transitional phrases, terms or words that do not preclude additional acts or structures. The present disclosure also contemplates other embodiments “comprising,” “consisting of” and “consisting essentially of,” the embodiments or elements presented herein, whether explicitly set forth or not.

The term “about” or “approximately” means within an acceptable error range for the particular value as determined by one of ordinary skill in the art, which depends in part on how the value is measured or determined, i.e., the limitations of the measurement system.

The term “biological fluid,” as used herein, refers to any bodily fluid or bodily fluid derivative in which the analyte can be measured. Non-limiting examples of a biological fluid include dermal fluid, interstitial fluid, plasma, blood, lymph, synovial fluid, cerebrospinal fluid, saliva, bronchoalveolar lavage, amniotic fluid, sweat, tears, or the like. In certain embodiments, the biological fluid is dermal fluid or interstitial fluid.

The term “reference electrode” as used herein, can refer to either reference electrodes or electrodes that function as both, a reference and a counter electrode. Similarly, the term “counter electrode,” as used herein, can refer to both, a counter electrode and a counter electrode that also functions as a reference electrode.

As used herein, the term “homogenous membrane” refers to a membrane including a single type of membrane polymer. As used herein, the term “multi-component membrane” refers to a membrane including two or more types of membrane polymers.

### Analyte Sensors & Sensor Configurations

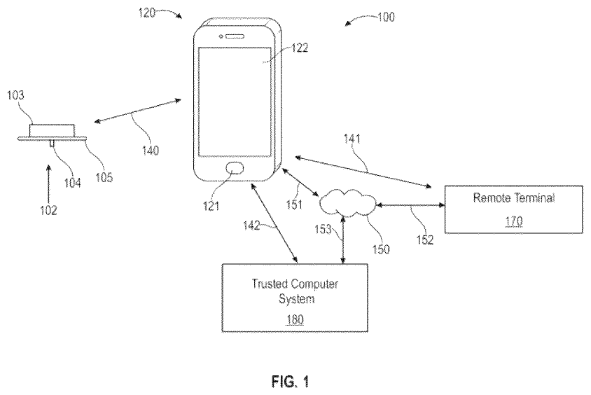

Before describing the analyte sensors of the present disclosure and their components in further detail, a brief overview of suitable in vivo analyte sensor configurations and sensor systems employing the analyte sensors will be provided so that the embodiments of the present disclosure can be better understood. FIG. 1 shows a diagram of an illustrative sensing system that can incorporate an analyte sensor of the present disclosure. As shown, sensing system 1 includes sensor control device 102 and reader device 120 that are configured to communicate with one another over a local communication path or link 140, which can be wired or wireless, uni- or bidirectional, and encrypted or non-encrypted. Reader device 120 can constitute an output medium for viewing analyte concentrations and alerts or notifications determined by sensor 104 or a processor associated therewith, as well as allowing for one or more user inputs, according to certain embodiments. Reader device 120 can be a multi-purpose smartphone or a dedicated electronic reader instrument. While only one reader device 120 is shown, multiple reader devices 120 can be present in certain embodiments. Reader device 120 can also be in communication with remote terminal 170 and/or trusted computer system 180 via communication path(s)/link(s) 141 and/or 142, respectively, which also can be wired or wireless, uni- or bi-directional, and encrypted or non-encrypted. Reader device 120 can also or alternately be in communication with network 150 (e.g., a mobile telephone network, the internet, or a cloud server) via communication path/link 151. Network 150 can be further communicatively coupled to remote terminal 170 via communication path/link 152 and/or trusted computer system 180 via communication path/link 153. Alternately, sensor 104 can communicate directly with remote terminal 170 and/or trusted computer system 180 without an intervening reader device 120 being present. For example, but not by the way of limitation, sensor 104 can communicate with remote terminal 170 and/or trusted computer system 180 through a direct communication link to network 150, according to certain embodiments, as described in U.S. Patent Application Publication 2011/0213225 and incorporated herein by reference in its entirety. Any suitable electronic communication protocol can be used for each of the communication paths or links, such as near field communication (NFC), radio frequency identification (RFID), BLUETOOTH® or BLUETOOTH® Low Energy protocols, WiFi, or the like. Remote terminal 170 and/or trusted computer system 180 can be accessible, according to certain embodiments, by individuals other than a primary user who have an interest in the user's analyte levels. Reader device 120 can include display 122 and optional input component 121. Display 122 can include a touch-screen interface, according to certain embodiments.

Sensor control device 102 includes sensor housing 103, which can house circuitry and a power source for operating sensor 104. Optionally, the power source and/or active circuitry can be omitted. A processor (not shown) can be communicatively coupled to sensor 104, with the processor being physically located within sensor housing 103 or reader device 120. Sensor 104 protrudes from the underside of sensor housing 103 and extends through adhesive layer 105, which is adapted for adhering sensor housing 103 to a tissue surface, such as skin, according to certain embodiments.

Sensor 104 is adapted to be at least partially inserted into a tissue of interest, such as within the dermal or subcutaneous layer of the skin. Sensor 104 can include a sensor tail of sufficient length for insertion to a desired depth in a given tissue. The sensor tail can include at least one working electrode. In certain configurations, the sensor tail can include an active area for detecting an analyte disposed upon the working electrode. A counter electrode can be present in combination with the at least one working electrode. Particular electrode configurations upon the sensor tail are described in more detail below.

The active area can be configured for monitoring a particular analyte, e.g., potassium, e.g., potassium ions. In certain embodiments, the active area can be configured for detecting two or more analytes.

In certain embodiments of the present disclosure, one or more analytes can be monitored in any biological fluid of interest such as dermal fluid, interstitial fluid, plasma, blood, lymph, synovial fluid, cerebrospinal fluid, saliva, bronchoalveolar lavage, amniotic fluid, or the like. In certain particular embodiments, analyte sensors of the present disclosure can be adapted for assaying dermal fluid or interstitial fluid to determine a concentration of one or more analytes in vivo. In certain embodiments, the biological fluid is interstitial fluid.

Referring still to FIG. 1, sensor 104 can automatically forward data to reader device 120. For example but not by the way of limitation, analyte concentration data (e.g., potassium ion concentrations) can be communicated automatically and periodically, such as at a certain frequency as data is obtained or after a certain time period has passed, with the data being stored in a memory until transmittal (e.g., every minute, five minutes, or other predetermined time period). In certain other embodiments, sensor 104 can communicate with reader device 120 in a non-automatic manner and not according to a set schedule. For example, but not by the way of limitation, data can be communicated from sensor 104 using RFID technology when the sensor electronics are brought into communication range of reader device 120. Until communicated to reader device 120, data can remain stored in a memory of sensor 104. Thus, a user does not have to maintain close proximity to reader device 120 at all times, and can instead upload data at a convenient time. In certain other embodiments, a combination of automatic and non-automatic data transfer can be implemented. For example, and not by the way of limitation, data transfer can continue on an automatic basis until reader device 120 is no longer in communication range of sensor 104.

An introducer can be present transiently to promote introduction of sensor 104 into a tissue. In certain illustrative embodiments, the introducer can include a needle or similar sharp. As would be readily recognized by a person skilled in the art, other types of introducers, such as sheaths or blades, can be present in alternative embodiments. More specifically, the needle or other introducer can transiently reside in proximity to sensor 104 prior to tissue insertion and then be withdrawn afterward. While present, the needle or other introducer can facilitate insertion of sensor 104 into a tissue by opening an access pathway for sensor 104 to follow. For example, and not by the way of limitation, the needle can facilitate penetration of the epidermis as an access pathway to the dermis to allow implantation of sensor 104 to take place, according to one or more embodiments. After opening the access pathway, the needle or other introducer can be withdrawn so that it does not represent a sharps hazard. In certain embodiments, suitable needles can be solid or hollow, beveled or non-beveled, and/or circular or non-circular in cross-section. In more particular embodiments, suitable needles can be comparable in cross-sectional diameter and/or tip design to an acupuncture needle, which can have a cross-sectional diameter of about 250 microns. However, suitable needles can have a larger or smaller cross-sectional diameter if needed for certain particular applications.

In certain embodiments, a tip of the needle (while present) can be angled over the terminus of sensor 104, such that the needle penetrates a tissue first and opens an access pathway for sensor 104. In certain embodiments, sensor 104 can reside within a lumen or groove of the needle, with the needle similarly opening an access pathway for sensor 104. In either case, the needle is subsequently withdrawn after facilitating sensor insertion.

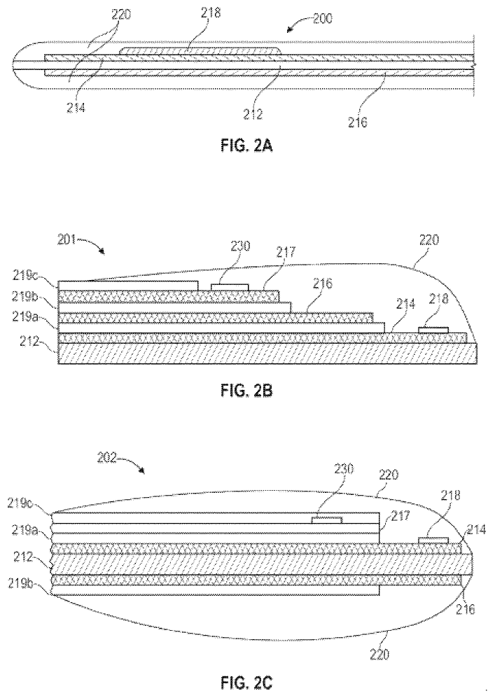

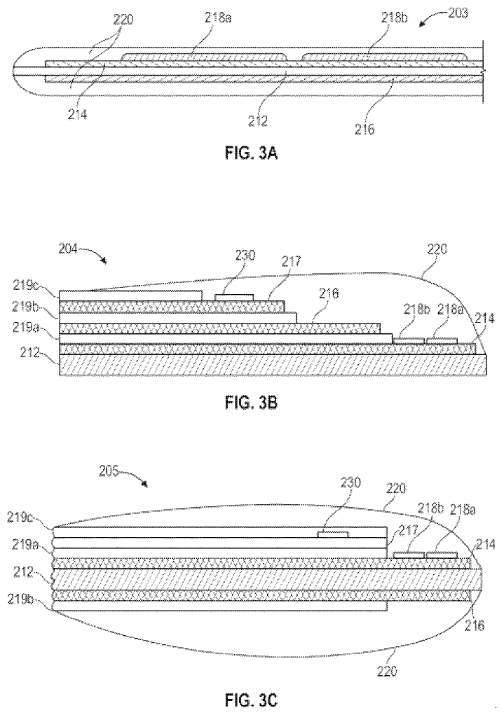

Sensor configurations featuring a single active area that is configured for the detection of a corresponding single analyte can employ two-electrode or three-electrode detection motifs, as described further herein in reference to FIGS. 2A-2C. Sensor configurations featuring two different active areas for detection of the same or separate analytes, either upon separate working electrodes or upon the same working electrode, are described separately thereafter in reference to FIGS. 3A-5C. Sensor configurations having multiple working electrodes can be particularly advantageous for incorporating two different active areas within the same sensor tail, since the signal contribution from each active area can be determined more readily.

When a single working electrode is present in an analyte sensor, three-electrode sensor configurations can include a working electrode, a counter electrode, and a reference electrode. Related two-electrode sensor configurations can include a working electrode and a second electrode, in which the second electrode can function as both a counter electrode and a reference electrode (i.e., a counter/reference electrode). The various electrodes can be at least partially stacked (layered) upon one another and/or laterally spaced apart from one another upon the sensor tail. Suitable sensor configurations can be substantially flat in shape or substantially cylindrical in shape or any other suitable shape. In any of the sensor configurations disclosed herein, the various electrodes can be electrically isolated from one another by a dielectric material or similar insulator.

FIG. 2A shows a diagram of an illustrative two-electrode analyte sensor configuration, which is compatible for use in the disclosure herein. As shown, analyte sensor 200 includes substrate 212 disposed between working electrode 214 and counter/reference electrode 216. Alternately, working electrode 214 and counter/reference electrode 216 can be located upon the same side of substrate 212 with a dielectric material interposed in between (configuration not shown). Active area 218 is disposed as at least one layer upon at least a portion of working electrode 214. Active area 218 can include multiple spots or a single spot configured for detection of an analyte, as discussed further herein.

Referring still to FIG. 2A, membrane 220 overcoats at least active area 218. In certain embodiments, membrane 220 can also overcoat some or all of working electrode 214 and/or counter/reference electrode 216, or the entirety of analyte sensor 200. One or both faces of analyte sensor 200 can be overcoated with membrane 220. Membrane 220 can include one or more polymeric membrane materials having capabilities of limiting analyte flux to active area 218 (e.g., membrane 220 is a mass transport limiting membrane having some permeability for the analyte of interest). According to the disclosure herein, and further described below, membrane 220 can be crosslinked with a branched crosslinker in certain particular sensor configurations. For example, but not by way of limitation, membrane 220 is crosslinked with a branched glycidyl ether. The composition and thickness of membrane 220 can vary to promote a desired analyte flux to active area 218, thereby providing a desired signal intensity and stability. Analyte sensor 200 can be operable for assaying an analyte by any of coulometric, amperometric, voltammetric, or potentiometric electrochemical detection techniques.

FIGS. 2B and 2C show diagrams of illustrative three-electrode analyte sensor configurations, which are also compatible for use in the disclosure herein. Three-electrode analyte sensor configurations can be similar to that shown for analyte sensor 200 in FIG. 2A, except for the inclusion of additional electrode 217 in analyte sensors 201 and 202 (FIGS. 2B and 2C). With additional electrode 217, counter/reference electrode 216 can then function as either a counter electrode or a reference electrode, and additional electrode 217 fulfils the other electrode function not otherwise accounted for. Working electrode 214 continues to fulfil its original function. Additional electrode 217 can be disposed upon either working electrode 214 or electrode 216, with a separating layer of dielectric material in between. For example, and not by the way of limitation, as depicted in FIG. 2B, dielectric layers 219a, 219b and 219c separate electrodes 214, 216 and 217 from one another and provide electrical isolation. Alternatively, at least one of electrodes 214, 216 and 217 can be located upon opposite faces of substrate 212, as shown in FIG. 2C. Thus, in certain embodiments, electrode 214 (working electrode) and electrode 216 (counter electrode) can be located upon opposite faces of substrate 212, with electrode 217 (reference electrode) being located upon one of electrodes 214 or 216 and spaced apart therefrom with a dielectric material.

Reference material layer 230 (e.g., Ag/AgCl) can be present upon electrode 217, with the location of reference material layer 230 not being limited to that depicted in FIGS. 2B and 2C. As with sensor 200 shown in FIG. 2A, active area 218 in analyte sensors 201 and 202 can include multiple spots or a single spot. Additionally, analyte sensors 201 and 202 can be operable for assaying an analyte by any of coulometric, amperometric, voltammetric or potentiometric electrochemical detection techniques.

Like analyte sensor 200, membrane 220 can also overcoat active area 218, as well as other sensor components, in analyte sensors 201 and 202, thereby serving as a mass transport limiting membrane. In certain embodiments, the additional electrode 217 can be overcoated with membrane 220. Although FIGS. 2B and 2C have depicted electrodes 214, 216 and 217 as being overcoated with membrane 220, it is to be recognized that in certain embodiments only working electrode 214 is overcoated. Moreover, the thickness of membrane 220 at each of electrodes 214, 216 and 217 can be the same or different. As in two-electrode analyte sensor configurations (FIG. 2A), one or both faces of analyte sensors 201 and 202 can be overcoated with membrane 220 in the sensor configurations of FIGS. 2B and 2C, or the entirety of analyte sensors 201 and 202 can be overcoated. Accordingly, the three-electrode sensor configurations shown in FIGS. 2B and 2C should be understood as being non-limiting of the embodiments disclosed herein, with alternative electrode and/or layer configurations remaining within the scope of the present disclosure.

FIG. 3A shows an illustrative configuration for sensor 203 having a single working electrode with two different active areas disposed thereon. FIG. 3A is similar to FIG. 2A, except for the presence of two active areas upon working electrode 214: first active area 218a and second active area 218b, which are responsive to the same or different analytes and are laterally spaced apart from one another upon the surface of working electrode 214. Active areas 218a and 218b can include multiple spots or a single spot configured for detection of each analyte. The composition of membrane 220 can vary or be compositionally the same at active areas 218a and 218b. First active area 218a and second active area 218b can be configured to detect their corresponding analytes at working electrode potentials that differ from one another, as discussed further below.

FIGS. 3B and 3C show cross-sectional diagrams of illustrative three-electrode sensor configurations for sensors 204 and 205, respectively, each featuring a single working electrode having first active area 218a and second active area 218b disposed thereon. FIGS. 3B and 3C are otherwise similar to FIGS. 2B and 2C and can be better understood by reference thereto. As with FIG. 3A, the composition of membrane 220 can vary or be compositionally the same at active areas 218a and 218b.

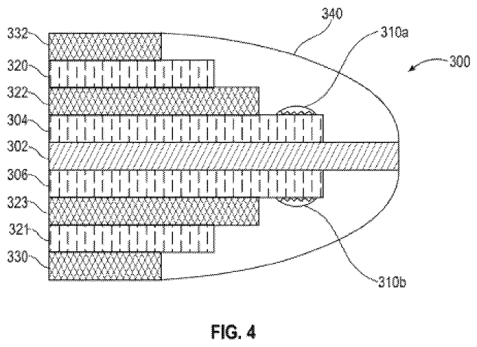

Illustrative sensor configurations having multiple working electrodes, specifically two working electrodes, are described in further detail in reference to FIGS. 4-SC. Although the following description is primarily directed to sensor configurations having two working electrodes, it is to be appreciated that more than two working electrodes can be incorporated through extension of the disclosure herein. Additional working electrodes can be used to impart additional sensing capabilities to the analyte sensors beyond just a first analyte and a second analyte, e.g., for the detection of a third and/or fourth analyte.

FIG. 4 shows a cross-sectional diagram of an illustrative analyte sensor configuration having two working electrodes, a reference electrode and a counter electrode, which is compatible for use in the disclosure herein. As shown, analyte sensor 300 includes working electrodes 304 and 306 disposed upon opposite faces of substrate 302. First active area 310a is disposed upon the surface of working electrode 304, and second active area 310b is disposed upon the surface of working electrode 306. Counter electrode 320 is electrically isolated from working electrode 304 by dielectric layer 322, and reference electrode 321 is electrically isolated from working electrode 306 by dielectric layer 323. Outer dielectric layers 330 and 332 are positioned upon reference electrode 321 and counter electrode 320, respectively. Membrane 340 can overcoat at least active areas 310a and 310b, according to various embodiments, with other components of analyte sensor 300 or the entirety of analyte sensor 300 optionally being overcoated with membrane 340. In certain embodiments, membrane 340 can be continuous but vary compositionally upon active area 310a and/or upon active area 310b in order to afford different permeability values for differentially regulating the analyte flux at each location. For example, different membrane formulations can be sprayed and/or printed onto the opposing faces of analyte sensor 300. Dip coating techniques can also be appropriate, particularly for depositing at least a portion of a bilayer membrane upon one of active areas 310a and 310b. In certain embodiments, membrane 340 can be the same or vary compositionally at active areas 310a and 310b. For example, but not by way of limitation, membrane 340 can include a bilayer overcoating active area 310a and be a homogeneous membrane overcoating active area 310b, or membrane 340 can include a bilayer overcoating active areas 310b and be a homogeneous membrane overcoating active area 310a. In certain embodiments, an analyte sensor can include more than one membrane 340, e.g., two or more membranes. For example, but not by way of limitation, an analyte sensor can include a membrane that overcoats the one or more active areas, e.g., 310a and 310b, and an additional membrane that overcoats the entire sensor as shown in FIG. 4.

Alternative sensor configurations having multiple working electrodes and differing from the configuration shown in FIG. 4 can feature a counter/reference electrode instead of separate counter and reference electrodes 320, 321, and/or feature layer and/or membrane arrangements varying from those expressly depicted. For example, and not by the way of limitation the positioning of counter electrode 320 and reference electrode 321 can be reversed from that depicted in FIG. 4. In addition, working electrodes 304 and 306 need not necessarily reside upon opposing faces of substrate 302 in the manner shown in FIG. 4.

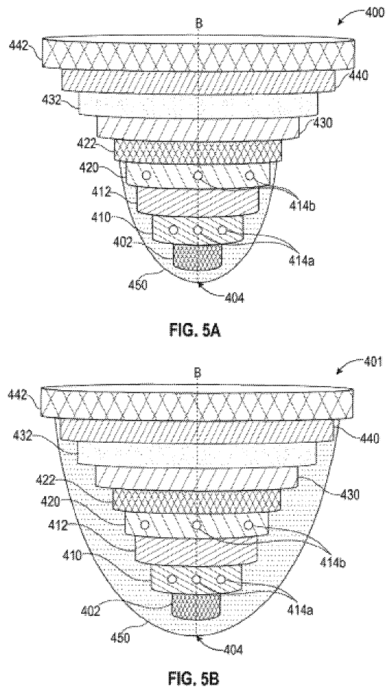

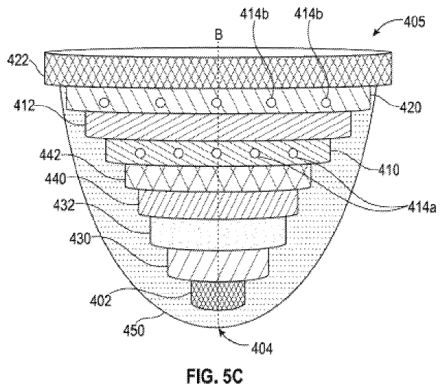

Although suitable sensor configurations can feature electrodes that are substantially planar in character, it is to be appreciated that sensor configurations featuring non-planar electrodes can be advantageous and particularly suitable for use in the disclosure herein. In particular, substantially cylindrical electrodes that are disposed concentrically with respect to one another can facilitate deposition of a mass transport limiting membrane, as described hereinbelow. FIGS. 5A-5C show perspective views of analyte sensors featuring two working electrodes that are disposed concentrically with respect to one another. It is to be appreciated that sensor configurations having a concentric electrode disposition but lacking a second working electrode are also possible in the present disclosure.

FIG. 5A shows a perspective view of an illustrative sensor configuration in which multiple electrodes are substantially cylindrical and are disposed concentrically with respect to one another about a central substrate. As shown, analyte sensor 400 includes central substrate 402 about which all electrodes and dielectric layers are disposed concentrically with respect to one another. In particular, working electrode 410 is disposed upon the surface of central substrate 402, and dielectric layer 412 is disposed upon a portion of working electrode 410 distal to sensor tip 404. Working electrode 420 is disposed upon dielectric layer 412, and dielectric layer 422 is disposed upon a portion of working electrode 420 distal to sensor tip 404. Counter electrode 430 is disposed upon dielectric layer 422, and dielectric layer 432 is disposed upon a portion of counter electrode 430 distal to sensor tip 404. Reference electrode 440 is disposed upon dielectric layer 432, and dielectric layer 442 is disposed upon a portion of reference electrode 440 distal to sensor tip 404. As such, exposed surfaces of working electrode 410, working electrode 420, counter electrode 430, and reference electrode 440 are spaced apart from one another along longitudinal axis B of analyte sensor 400.

Referring still to FIG. 5A, first active areas 414a and second active areas 414b, which are responsive to different analytes or the same analyte, are disposed upon the exposed surfaces of working electrodes 410 and 420, respectively, thereby allowing contact with a fluid to take place for sensing. Although active areas 414a and 414b have been depicted as three discrete spots in FIG. 5A, it is to be appreciated that fewer or greater than three spots, including a continuous layer of active area, can be present in alternative sensor configurations.

In FIG. 5A, sensor 400 is partially coated with membrane 450 upon working electrodes 410 and 420 and active areas 414a and 414b disposed thereon. FIG. 5B shows an alternative sensor configuration in which the substantial entirety of sensor 401 is overcoated with membrane 450. Membrane 450 can be the same or vary compositionally at active areas 414a and 414b. For example, membrane 450 can include a bilayer overcoating active areas 414a and be a homogeneous membrane overcoating active areas 414b.

It is to be further appreciated that the positioning of the various electrodes in FIGS. 5A and 5B can differ from that expressly depicted. For example, the positions of counter electrode 430 and reference electrode 440 can be reversed from the depicted configurations in FIGS. 5A and 5B. Similarly, the positions of working electrodes 410 and 420 are not limited to those that are expressly depicted in FIGS. 5A and 5B. FIG. 5C shows an alternative sensor configuration to that shown in FIG. 5B, in which sensor 405 contains counter electrode 430 and reference electrode 440 that are located more proximal to sensor tip 404 and working electrodes 410 and 420 that are located more distal to sensor tip 404. Sensor configurations in which working electrodes 410 and 420 are located more distal to sensor tip 404 can be advantageous by providing a larger surface area for deposition of active areas 414a and 414b (five discrete sensing spots illustratively shown in FIG. 5C), thereby facilitating an increased signal strength in some cases. Similarly, central substrate 402 can be omitted in any concentric sensor configuration disclosed herein, wherein the innermost electrode can instead support subsequently deposited layers.

### Sensor Chemistries & Electrochemical Detection Methods

As discussed in detail above, the analyte sensor of the present disclosure comprises a potassium responsive active area; wherein the potassium responsive active area comprises a potassium selective transport layer forming an outer surface of the potassium responsive active area, wherein the potassium selective transport layer comprises a polymer, a potassium ionophore, a plasticizer, and an electrolyte. Optionally, the sensor further comprises an electrolyte gel layer disposed between the potassium selective transport layer and the portion of the outer surface of the first working electrode, although as discussed above, a key aspect of some embodiments of the invention is that the presence of such a layer is not required when the method of the present invention is used.

The potassium responsive active area serves to detect the presence and concentration of potassium ions in a fluid in which the analyte sensor is disposed. When the analyte sensor is disposed within a potassium ion containing fluid, potassium ions migrate into the potassium selective transport layer by reversibly binding to the potassium ionophore. This process occurs upon reduction of a transition metal ion at the electrode. The number of potassium ions that migrate from the fluid into the potassium selective transport layer is proportional to the concentration of potassium ions within the fluid in which the sensor is disposed. Hence, a higher concentration of potassium ions within the fluid will cause a higher concentration of potassium ions within the potassium selective transport layer. The purpose of the potassium ionophore is to allow the potassium ions to enter the potassium selective transport layer. Potassium ions in aqueous solution are hydrophilic and so typically do not migrate into a hydrophobic polymer layer such as PVC. Ionophores are typically hydrophobic molecules and so can be effectively dispersed within the polymer of the potassium selective transport layer. The reversible binding of the ionophore to the potassium ions causes the potassium ions to be in a suitably hydrophobic form allowing them to migrate into the polymer layer.

On application of a potential to the working electrode, a transition metal ion present in the portion of the first working electrode is reduced to a lower oxidation state. This causes a charge imbalance at the working electrode due to the presence of the anionic counterions. In order to balance charges at the working electrode, anions migrate from the portion of the first working electrode to the electrolyte gel layer. For example, where the sensor comprises silver and silver chloride in the portion of the first working electrode, silver chloride (AgCl) is reduced to elemental silver, and the chloride ion migrates into the electrolyte gel layer. Similarly, where the portion of the first working electrode comprises a mixture of osmium (II) ions and osmium (III) ions (such as osmium ions in complex form), an osmium (III) ion is reduced on application of electrical potential to the first working electrode to an osmium (II) ion and the counterion of the complex migrates into the electrolyte gel layer to balance the charges. The flow of anions into the electrolyte gel layer causes a charge imbalance in the electrolyte gel layer. In order to balance charges in the electrolyte gel layer, anions flow from the electrolyte gel layer into the potassium selective transport layer and thus cause an imbalance of charges in the potassium selective transport layer. In order to counteract this charge imbalance in the potassium selective transport layer, potassium ions from the fluid in which the sensor is disposed are drawn into the potassium selective transport layer. If the electrolyte gel layer is not present, ions flow directly from the electrode portion into the potassium selective transport layer.

The electrode obtains a signal indicative of the flow of potassium ions from the fluid in which the sensor is disposed into the potassium selective transport layer. In some embodiments, the signal is detected as a current. The amount of potassium ions that are able to flow into the potassium selective transport layer from the biological fluid in which the sensor is disposed is the limiting step in the ion flow cascade. Accordingly, the magnitude of the current detected is positively correlated with how many potassium ions are able to flow into the potassium selective transport layer from the fluid in which the sensor is disposed. As discussed above, how many potassium ions are able to enter the potassium selective transport layer is correlated with the concentration of potassium ions in the fluid in which the sensor is disposed. The signal detected is thus indicative of the potassium ion concentration in the fluid in which the sensor is disposed.

The signal detected is also indicative of the extent of the reduction that occurs at the first working electrode. The strength of the signal will be correlated to the degree of the reduction that occurs at the electrode. The degree of the reduction that occurs at the electrode is correlated to the number of potassium ions that have migrated from the fluid into the potassium selective transport layer (which is correlated to the concentration of potassium ions in the fluid, as discussed above) in order to balance charges. Since the migration of potassium ions into the potassium selective transport layer is the limiting factor in the ion flow cascade, the amount of potassium ions that are able to migrate into the potassium selective transport layer thus determines the degree of reduction that is able to occur at the electrode. Since the amount of potassium ions that are able to migrate from the fluid into the potassium selective transport layer is correlated with the concentration of potassium ions in the biological fluid, the strength of the signal obtained at the electrode is thus correlated to the concentration of potassium ions within the fluid in which the sensor is disposed. The sensor signal can thus indicate the potassium ion concentration of the fluid in which the sensor is disposed.

Once the processes described above have occurred, the transition metal salt can be recovered at the first working electrode by the application of electrical potential to the electrode causing oxidation of the reduced species. For example, in the systems described above, silver atoms may be oxidised to Ag⁺ ions and osmium (II) complex ions may be oxidised to osmium (III) complex ions. This process causes anions to migrate from the electrolyte gel layer back into the portion of the first working electrode in order to balance charges and provide the salt silver chloride or the osmium (III) salt. This process in turn causes anions to migrate from the potassium selective transport layer into the electrolyte gel layer in order to balance charges in the electrolyte gel layer. To balance charges in the potassium selective transport layer, the potassium ions migrate from this layer back into the surrounding fluid in which the sensor is disposed. Where an electrolyte gel layer is not present, ions flow directly from the potassium selective transport layer into the portion of the working electrode. The electrode then obtains a signal indicative of the flow of potassium ions from the potassium selective transport layer back into the surrounding fluid. In some embodiments, the signal is current. The measured current is indicative of the concentration of potassium ions in the fluid in which the sensor is disposed, as discussed above. This is because the amount of potassium ions expelled from the potassium selective transport layer is equivalent (or at least very similar) to the amount of potassium ions that were originally drawn into the layer during the reduction step, which as discussed above, is correlated with the concentration of potassium ions in the fluid in which the sensor is disposed.

The degree of oxidation that occurs at the electrode is correlated with the number of potassium ions within the potassium selective transport layer. Since the number of potassium ions within the potassium selective transport layer is correlated with the concentration of ions within the fluid being analysed, the degree of oxidation is thus correlated with the concentration of potassium ions in the fluid in which the sensor is disposed. An electrode signal can thus also be obtained for the oxidation process described above that occurs at the electrode, where the signal is indicative of the concentration of potassium ions in the fluid being analysed.

The sensors of the present invention can operate continuously due to the reversibility of potassium ions being able to enter and exit the potassium selective transport layer and the corresponding oxidation/reduction processes that occur at the electrode thus generating a signal indicative of potassium ion concentration in the fluid being analysed. Potassium ions can enter the potassium selective transport layer so that a signal is generated which is indicative of the potassium ion concentration of the fluid being analysed. Since the potassium ions can then subsequently be caused to migrate out of the potassium selective transport layer, the sensor can then be used again in another measurement cycle in which potassium ions once again migrate into the potassium selective transport layer. The sensor can thus continuously monitor the concentration of potassium ions in a fluid in which the sensor is disposed, and changes in potassium ion concentration in the fluid over time.

Where an electrolyte gel layer is included in the sensors of the disclosure, the inclusion of such a layer is useful for extending the lifetime of the sensor. This is believed to be because the gel layer provides a reservoir of anions (from the electrolyte) that are able to migrate into the potassium selective transport layer as necessary, or into the portion of the working electrode as necessary. The electrolyte gel layer can also accept anions migrating from the potassium selective transport layer when oxidation occurs at the electrode. The electrolyte gel layer has also been found useful for providing a favourable medium to facilitate the reduction and oxidation reactions at the electrode surface. However, as discussed above, the inclusion of an electrolyte gel layer adds complexity and cost to processes for manufacturing the sensors. Accordingly, it is desirable to omit such a layer if a sensor can still be provided with sufficient functionality and lifetime. As discussed above, surprisingly, where the method of the invention is used, it has been found that omission of the electrolyte gel layer is possible and that the sensor can still be used to provide good detection of potassium in in vivo biological fluids.

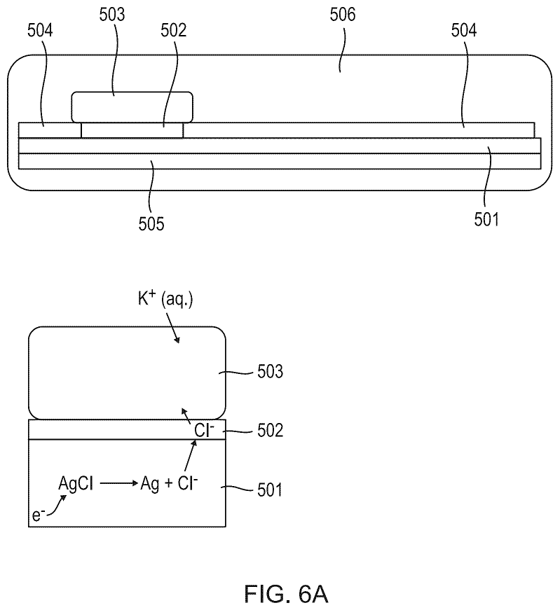

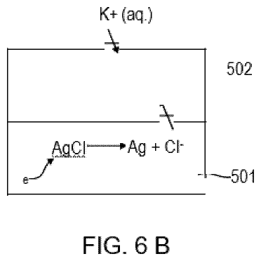

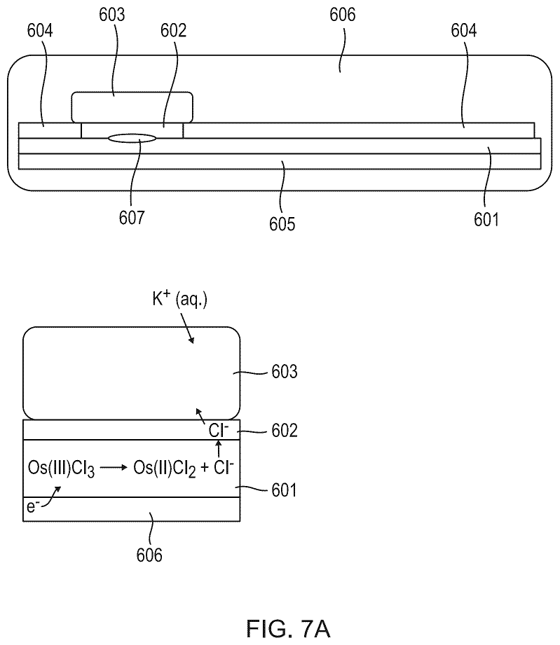

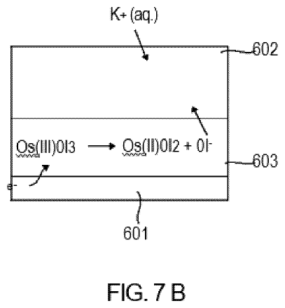

FIGS. 6 and 7 depict example sensor chemistries and configurations of analyte sensors of the present disclosure. FIGS. 6a and 7a show embodiments that employ a gel layer and FIGS. 6b and 7b show embodiments without a gel layer.

In FIG. 6a, the first working electrode is a silver/silver chloride electrode layer 501. Disposed adjacent to this electrode layer is the electrolyte gel layer 502. Disposed adjacent to the electrolyte gel layer is the potassium selective transport layer 503 which in this embodiment comprises PVC, plasticizer, electrolyte and potassium ionophore. The electrolyte gel layer 502 and potassium selective transport layer 503 together are the potassium responsive active areas. It can be seen that part of the silver/silver chloride layer 501 is directly exposed to the electrolyte gel layer 502, whilst part of the silver/silver chloride layer 501 extends further along the length of the working electrode with the dielectric layer 504. A PET electrode support layer 505 is adjacent to the Ag/AgCl layer 501. The working electrode is surrounded by a mass transport limiting membrane 506, as discussed in further detail below. The mass transport limiting membrane 506 limits the contact of the potassium responsive active area with the surrounding fluid in which the sensor is disposed, but is at least partially permeable to potassium ions to permit the potassium ions to contact the potassium responsive active area.

FIG. 7a shows an alternative configuration to the sensor configuration shown in FIG. 6a. In the configuration depicted in FIG. 7a, the silver/silver chloride electrode layer is replaced by a carbon electrode 601. On a portion of the outer surface of the carbon electrode 601 is an outer electrode portion 607 comprising a complex of osmium (II)/osmium (III) ions which has the potassium selective transport layer disposed upon it. A dielectric layer 604 is disposed on other portions of the carbon electrode 601. The outer electrode portion 607 comprises a redox polymer comprising a polymer backbone grafted to a ligand of the osmium complex (in the figure, the redox polymer is not depicted, and the osmium ions are depicted as simple osmium chloride salts). The electrolyte gel layer 602 is disposed adjacent to the outer electrode portion 607 and the potassium selective transport layer 603 is disposed adjacent to the electrolyte gel layer 602. A PET electrode support layer 605 is adjacent to the carbon electrode 601. The working electrode is surrounded by a mass transport limiting membrane 606.

In FIG. 6b, the first working electrode is a silver/silver chloride electrode layer 501. Disposed adjacent to the silver/silver chloride layer is the potassium selective transport layer 502 which in this embodiment comprises PVC, plasticizer, electrolyte and potassium ionophore. The potassium selective transport layer 502 forms the potassium responsive active area. Whilst it is not shown in the figure, part of the silver/silver chloride layer is directly exposed to the potassium selective transport layer 502, whilst part of the silver/silver chloride layer 501 extends further along the length of the working electrode directly adjacent to a dielectric layer. A PET electrode support layer is adjacent to the Ag/AgCl layer 501. The working electrode may be surrounded by a mass transport limiting membrane, as discussed in further detail below. The mass transport limiting membrane limits the contact of the potassium responsive active area with the surrounding fluid in which the sensor is disposed, but is at least partially permeable to potassium ions to permit the potassium ions to contact the potassium responsive active area.

FIG. 7b shows an alternative configuration to the sensor configuration shown in FIG. 6b. In the configuration depicted in FIG. 7b, the silver/silver chloride electrode layer is replaced by a carbon electrode 601. On a portion of the outer surface of the carbon electrode is an outer electrode portion 603 comprising a complex of osmium (II)/osmium (III) ions which has the potassium selective transport layer 602 disposed upon it. The outer electrode portion comprises a redox polymer comprising a polymer backbone grafted to a ligand of the osmium complex (in the figure, the redox polymer is not depicted, and the osmium ions are depicted as simple osmium chloride salts).

In addition to step (a) of providing an analyte sensor, the methods of the present invention comprise a step (b) of applying a potential to the first working electrode and a step (c) of obtaining a signal indicative of oxidation and/or reduction of the transition metal or transition metal salt of the first working electrode; wherein the signal is indicative of the concentration of potassium ions in the fluid.

As discussed above, in some embodiments, the methods of the invention involve applying steps of potential that alternate between two different values; and measuring the resultant current. The two different values are selected to correspond to respective oxidation and reduction at the working electrode. For example, a value may be chosen to correspond to electrochemical reduction of AgCl to silver metal, and the other value selected to electrochemically oxidize silver metal to form silver chloride. The resultant current is the obtained sensor signal and is indicative of reduction/oxidation that has occurred at the working electrode. The potassium concentration in the potassium selective transport layer and thus fluid in which the sensor is disposed can be determined from this measurement.

The specifics of how to carry out the above described method steps and obtain a signal will be familiar to a person skilled in the art given the benefit of the present disclosure. In some embodiments, the methods of the invention also comprise a step of determining the concentration of potassium ions in the fluid from the signal obtained. Methods of determining the potassium ion concentration from the obtained sensor signal will be apparent to those skilled in the art given the benefit of the present disclosure.

In the methods described above, it is also to be appreciated that the sensitivity (output current) of the analyte sensors toward each analyte can be varied by changing the coverage (area or size) of the active areas, the area ratio of the active areas with respect to one another, the identity, thickness and/or composition of a mass transport limiting membrane overcoating the active areas (as discussed in further detail below). Variation of these parameters can be conducted readily by one having ordinary skill in the art once granted the benefit of the disclosure herein.

### Mass Transport Limiting Membranes

In certain embodiments, the analyte sensors disclosed herein further include a membrane permeable to an analyte that overcoats at least an active area present on a working electrode of the analyte sensor.

In certain embodiments, a membrane overcoating an analyte-responsive active area can function as a mass transport limiting membrane and/or to improve biocompatibility. A mass transport limiting membrane can act as a diffusion-limiting barrier to reduce the rate of mass transport of the analyte. For example, but not by way of limitation, limiting access of an analyte, such as potassium, to the analyte-responsive active area with a mass transport limiting membrane can aid in avoiding sensor overload (saturation), thereby improving detection performance and accuracy. In certain embodiments, the mass transport limiting membrane can be homogeneous and can be single-component (contain a single membrane polymer). Alternatively, the mass transport limiting membrane can be multi-component (contain two or more different membrane polymers).

In certain embodiments, a multi-component membrane can be present as a bilayer membrane or as a homogeneous admixture of two or more membrane polymers. A homogeneous admixture can be deposited by combining the two or more membrane polymers in a solution and then depositing the solution upon a working electrode.

In certain embodiments, the composition of the mass transport limiting membrane disposed upon an analyte sensor that has two active areas can be the same or different where the mass transport limiting membrane overcoats each active area.

In certain embodiments, a mass transport limiting membrane can include a polyvinyl pyridine, a polyvinylimidazole, a copolymer of vinyl pyridine and styrene, a polyurethane or a polyether urethane, or a chemically related material, or membranes that are made of silicone, and the like. In certain embodiments, a membrane, e.g., a single-component membrane, can include a polyvinylpyridine. In certain embodiments, a membrane, e.g., a single-component membrane, can include a copolymer of vinylpyridine and styrene.

A suitable copolymer of vinyl pyridine and styrene can have a styrene content ranging from about 0.01% to about 50% mole percent, or from about 0.05% to about 45% mole percent, or from about 0.1% to about 40% mole percent, or from about 0.5% to about 35% mole percent, or from about 1% to about 30% mole percent, or from about 2% to about 25% mole percent, or from about 5% to about 20% mole percent. Substituted styrenes can be used similarly and in similar amounts.

A suitable copolymer of vinyl pyridine and styrene can have a weight average molecular weight of 5 kDa or more, or about 10 kDa or more, or about 15 kDa or more, or about 20 kDa or more, or about 25 kDa or more, or about 30 kDa or more, or about 40 kDa or more, or about 50 kDa or more, or about 75 kDa or more, or about 90 kDa or more, or about 100 kDa or more. In non-limiting examples, a suitable copolymer of vinyl pyridine and styrene can have a weight average molecular weight ranging from about 5 kDa to about 150 kDa, or from about 10 kDa to about 125 kDa, or from about 15 kDa to about 100 kDa, or from about 20 kDa to about 80 kDa, or from about 25 kDa to about 75 kDa, or from about 30 kDa to about 60 kDa.

In certain other embodiments, a membrane polymer overcoating one or more active areas can be crosslinked with a branched crosslinker including three or more crosslinkable groups, such as polyethyleneglycol tetraglycidyl ether, which can decrease the amount of extractables obtainable from the mass transport limiting membrane, as referenced above. In certain embodiments, the mass transport limiting membrane can include polyvinyl pyridine or a copolymer of vinyl pyridine and styrene crosslinked with a branched glycidyl ether crosslinker including three crosslinkable groups, such as polyethylene glycol tetraglycidyl ether. In certain embodiments, the epoxide groups of the polyethylene glycol tetraglycidyl ether can form a covalent bond with pyridine or an imidazole via epoxide ring opening resulting in a hydroxyalkyl group bridging a body of the crosslinker to the heterocycle of the membrane polymer.

Polydimethylsiloxane (PDMS) can be incorporated in any of the mass transport limiting membranes disclosed herein.

### Methods of Manufacture

The present disclosure further provides methods for manufacturing the analyte sensors of the present disclosure. Suitable methods for manufacturing analyte sensors disclosed herein will be apparent to those of skill in the art given the benefit of the present disclosure.

In some embodiments, the manufacturing process starts by screen printing electrodes such as screen printing silver/silver chloride electrodes using techniques known in the art. If desired to include, an electrolyte gel layer can then be formed onto the exposed electrode (e.g., silver/silver chloride) area (as defined by the dielectric layer) by photopolymerizing monomers and crosslinking agents in a buffer with electrolyte ions. If the electrolyte gel layer is not included, steps such as this are omitted. The electrodes can then be dipped into a solution mixture of the polymer, plasticizer, electrolyte and ionophore to form the potassium selective layer. If desired, a membrane composition can then be cured on top of the sensor to form a mass transport limiting membrane.

### EXAMPLES

Examples 1 to 3 described below are included to demonstrate that sensors of the present disclosure with no electrolyte gel layer can function and detect the presence of potassium in biological fluids when a variety of electrochemical detection techniques are used.

However, the methods used in these examples do not involve chronoamperometry using the short potential durations as shown in the later examples. These examples are included to show that the sensors without an electrolyte gel layer present, and without use of chronoamperometry using the short potential durations, can detect potassium, but that the sensors have low sensor lifetime which limits their suitability for use in in vivo potassium detection methods.

### Example 1

A potassium sensor was manufactured. This potassium sensor does not comprise an electrolyte gel layer.

The potassium selective transport layer comprised 3% by weight PVC dissolved in an NPOE plasticizer solution. The PVC was dissolved in the plasticizer at a temperature of 120° C. The potassium ionophore was valinomycin which was included in the layer at a concentration of 10 mM. The layer also comprised tetraoctylammonium tetrakis (pentafluorophenyl) borate (TOATB) as the electrolyte, which was included in a concentration of 10 mM.

The first working electrode comprised a silver and silver chloride electrode layer.

The silver/silver chloride electrodes were screen printed before being dipped into PVC solution containing the potassium ionophore and other components of the potassium selective transport layer described above.

The sensor was then used to detect potassium concentration in a voltametric method in which electric potential was applied to the working electrode. The potential was varied at a scan rate of 5 mV/s between 0.5 V and −0.8 V with a CHI1040C potentiostat. The electrochemical cell used was a 3-electrode configuration with a screen printed carbon counter electrode and a screen printed silver/silver chloride reference electrode. The resulting current was recorded and plotted as a voltammogram. The peak current was used as the sensor signal to determine potassium concentration.

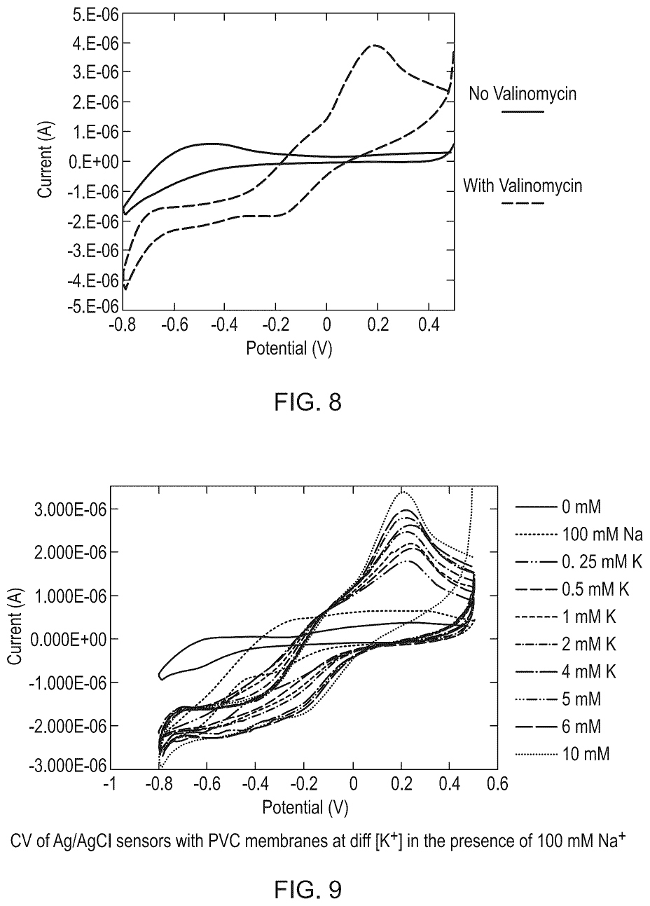

FIG. 8 compares the sensor responses in the presence and absence of valinomycin in the PVC polymer layer tested in a 20 mM tris buffer solution (pH=7.5, temperature=33° C.) containing 100 mM NaCl and 10 mM KCl. The result clearly shows that in the absence of valinomycin, the sensor current was much lower than that in the presence of valinomycin.

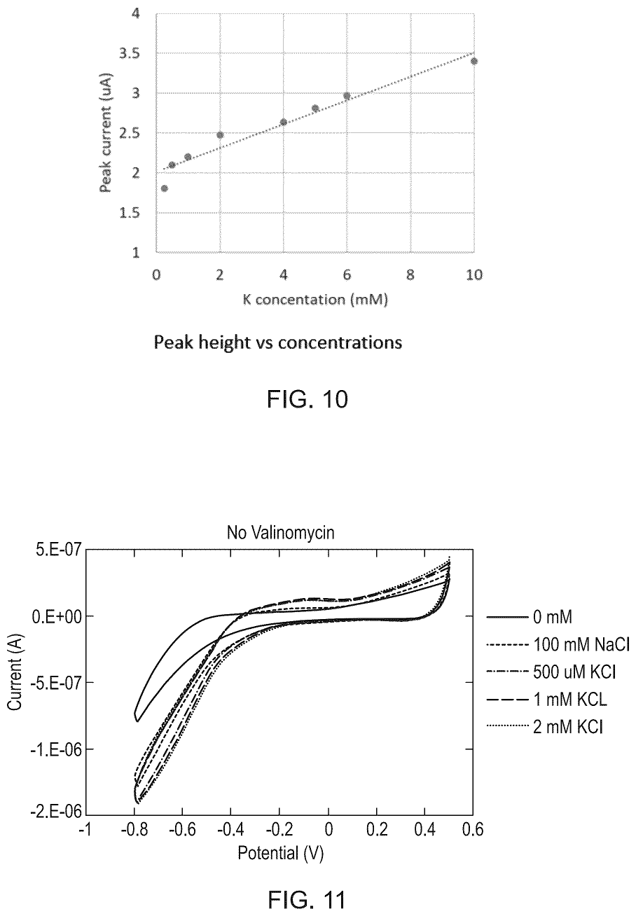

The experiment was repeated several times at various potassium concentrations with a sensor having valinomycin in the PVC layer. The resulting voltammograms of this experiment are shown in FIG. 9. It can be seen both the cathodic and anodic peak currents increased with increasing potassium concentrations. FIG. 10 plots the anodic peak current versus potassium concentration. A linear relationship was obtained within the concentration range from 1 to 10 mM. The peak current is thus indicative of the potassium concentration of the fluid.

Another comparative experiment was also carried out with no valinomycin present in the potassium selective transport layer. The results of this experiment are shown in FIG. 11. In FIG. 11, it can be seen that where the potassium selective transport layer does not comprise valinomycin, the peak current obtained was very similar and remained at very low levels at different potassium or sodium ion concentrations. This indicates that the sensor does not work where the potassium ionophore is not present in the layer.

### Example 2

A group of four similar sensors to the sensor used in Example 1 were manufactured using the same manufacturing methods.

The first working electrode comprised a silver and silver chloride electrode layer.

The potassium selective transport layer comprised 20% by weight PVC in NPOE, along with 10 mM of the same electrolyte as Example 1, and 10 mM of valinomycin. One sensor was also made with no valinomycin in this layer.

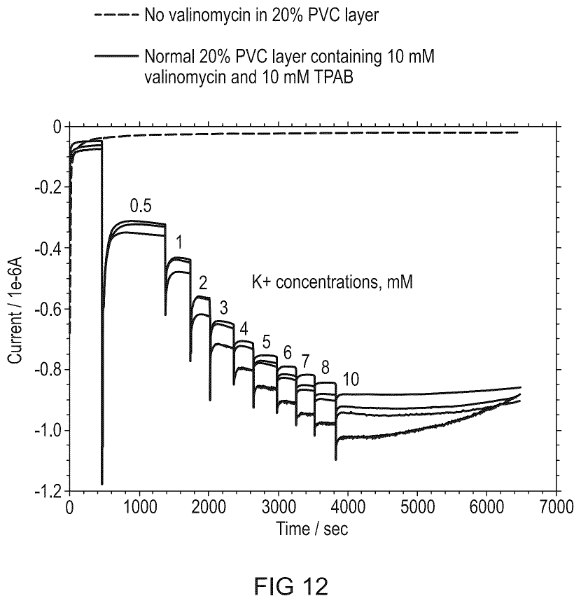

With the same electrochemical cell configuration, a conventional amperometry experiment was carried out where a constant potential at E=−200 mV was applied to the working electrodes of these sensors. The resulting current was recorded continuously when different amounts of KCl was added to a 20 mM tris buffer solution with 140 mM NaCl at 33° C. The results shown in FIG. 12 indicate that the current increases as the potassium concentration increased when valinomycin was included in the potassium selective transport layer. In the absence of valinomycin, the sensor current remained constant when potassium concentration increased. The sensor can thus be used to determine potassium concentration in a biological fluid.

### Example 3

A chronoamperometric method was also carried out with potential steps alternating between E=200 mV and E=−200 mV for 15 minutes each. The method does not involve pulse amperometry and has a longer duration of the potential steps (i.e., 15 minutes each) when compared to the methods of the invention.

To compare and demonstrate the advantage of this method, constant potentials at E=200 mV and E=−200 mV were also tested. The sensors used were all manufactured as the same way as in Example 2.

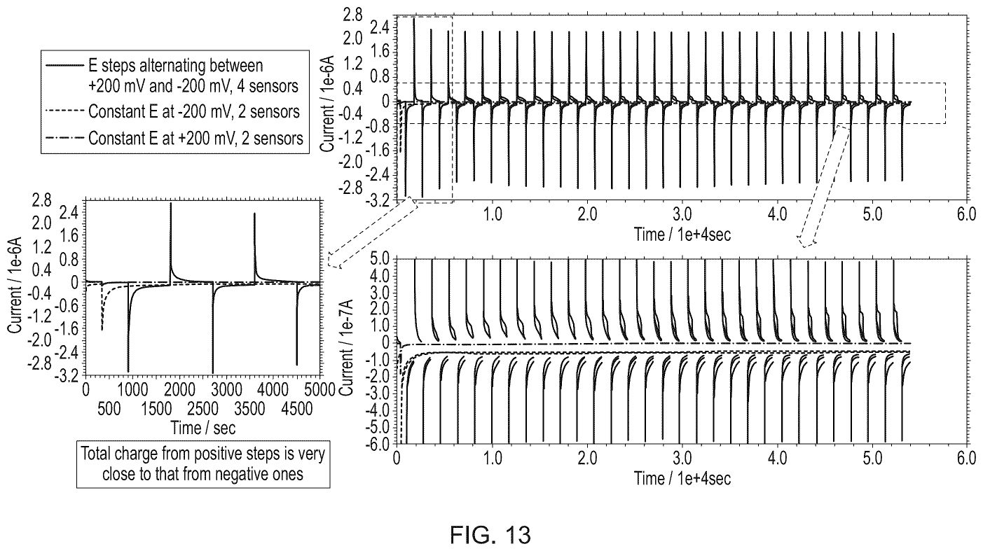

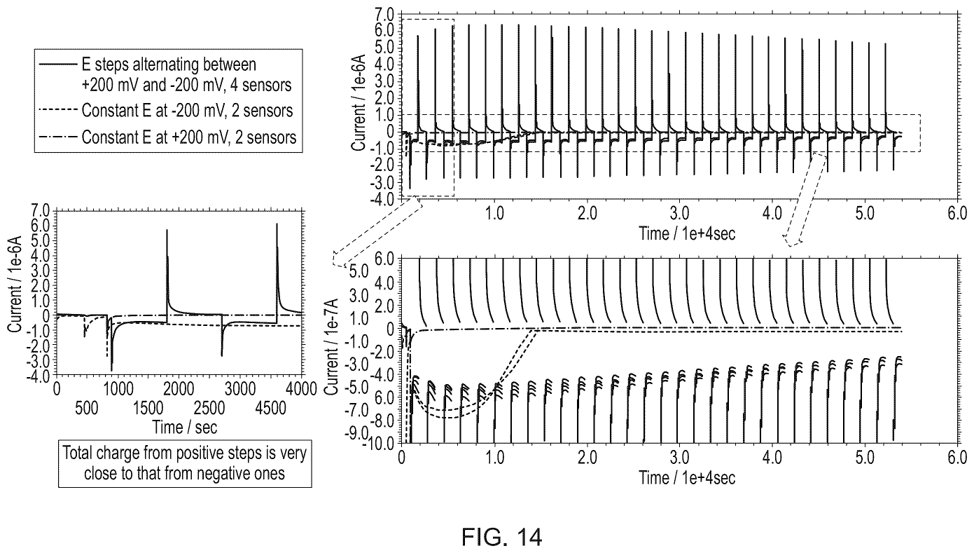

A first experiment, shown in FIG. 13, was carried out in a fluid containing 20 mM tris buffer and 140 mM NaCl without any potassium present. A second experiment, shown in FIG. 14, was then carried out in 20 mM tris buffer and 140 mM NaCl and 4 mM KCl. Both experiments involved 3 types of potential profiles applied to the sensors: alternating between +200 and −200 mV, constant at +200 mV, and constant at −200 mV. The results from these 3 different potential profiles are examined and analysed as below:

At constant 200 mV, the sensors in both experiments showed no response. This result was expected. At this potential, silver metal can be oxidized to generate positive charge at the working electrode surface. However, in order for the current to flow, some positive ions need to be expelled out from the organic PVC layer into the aqueous fluid. Since all the components in this layer are hydrophobic, their migration into the aqueous phase is prohibited. As a result, no current can flow under such condition.

At constant −200 mV, the result was similar to that from Example 2. Silver chloride can be reduced at the electrode surface under this condition. The extra negative charge from the generation of chloride anion drives the intake of potassium cations from the aqueous solution into the PVC layer because of the presence of valinomycin, resulting in a continuous current flow as shown in the trace of FIG. 14. The current is much smaller when the aqueous solution contains no potassium ions, as shown in the case of FIG. 13. It is notable that this current shown in FIG. 14 lasted about 3 hours and then decreased rapidly to essentially zero. This is because all the silver chloride present on the exposed electrode surface was reduced and the sensors stopped working.

To extend the sensor life, alternating potentials between +200 and −200 mV were applied to the working electrode. At these two potentials, the same electrode reactions and ion migrations described above can be applied here. However, the difference here is that at +200 mV, the current can flow because of the presence of potassium ions in the PVC layer migrated from the previous potential period at −200 mV. The silver chloride reduced at −200 mV can thus be recovered after the potential is switched to +200 mV. The result in FIG. 14 shows the extension of sensor life. The next example further improves on sensor performance in this respect.

### Example 4

Whilst the sensors used in examples 1, 2 and 3 were able to detect potassium concentration, the sensor lifetime was found to be relatively short. Sensors of the present invention comprising an additional hydrogel layer were thus manufactured to try and address this problem. The first working electrode comprised a silver and silver chloride electrode layer.

A hydrogel gel layer was then formed onto this working electrode according to the following procedure:
(1) Prepare the monomer solution by mixing 97 mol % N, N′-dimethylacrylamide and 3 mol % ethylene glycol dimethacrylate (EGDMA).
(2) Prepare the aqueous electrolyte solution containing 100 mM LiCl, 1 mM tetraoctylammonium chloride and 1 mM HEPES buffer with pH=8.0.
(3) UV polymerization: Mix the monomer solution and the aqueous solution at a ratio of 20/80. Transfer about 0.1 to 0.5 μL of such mixture onto the working electrode and expose the mixture to UV light at 254 nm wavelength for 5 minutes.

The potassium selective transport layer was then prepared with the same composition and procedure as described in Examples 2 and 3.

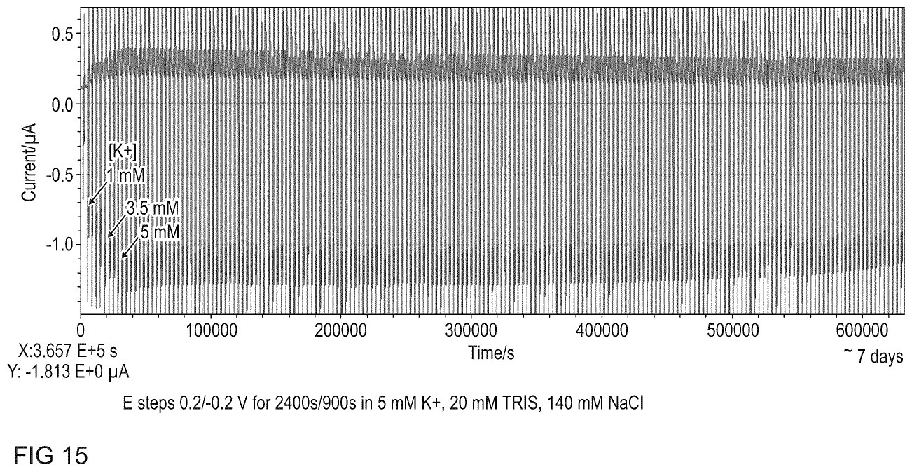

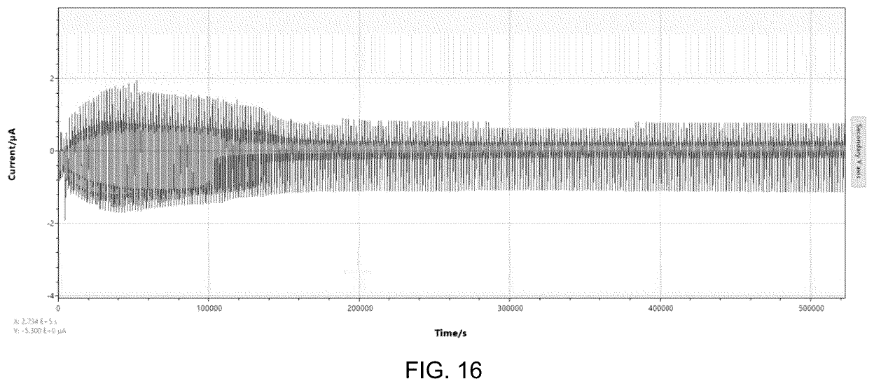

The sensor was used in a chronoamperometric method with potential steps alternating at +200 and −200 mV for 2400 seconds and 900 seconds, respectively, in a fluid containing 5 mM potassium ion, 20 mM TRIS and 140 mM NaCl at pH 7.5 and 33° C. The results shown in FIG. 15 demonstrate that chronoamperometry can be used to determine potassium concentration in a fluid in which the sensor is disposed. It can also be seen that the sensor current remained stable at 5 mM potassium for a duration of 7 days, indicating a significant improvement in sensor lifetime in the presence of the hydrogel layer under this potential step profile.

### Example 5

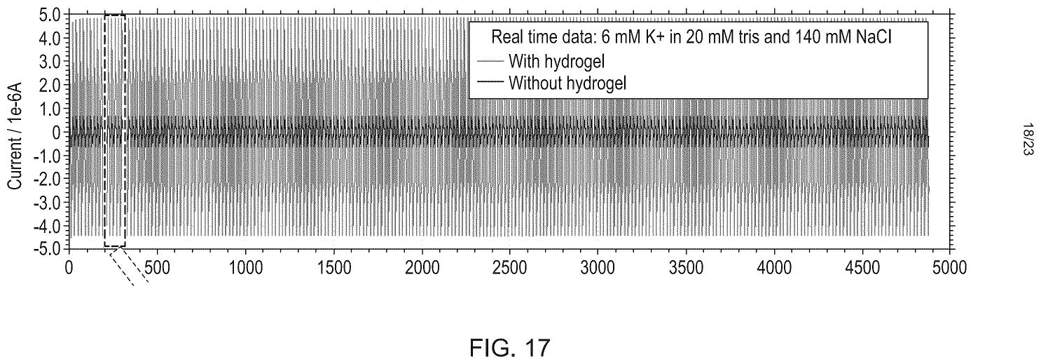

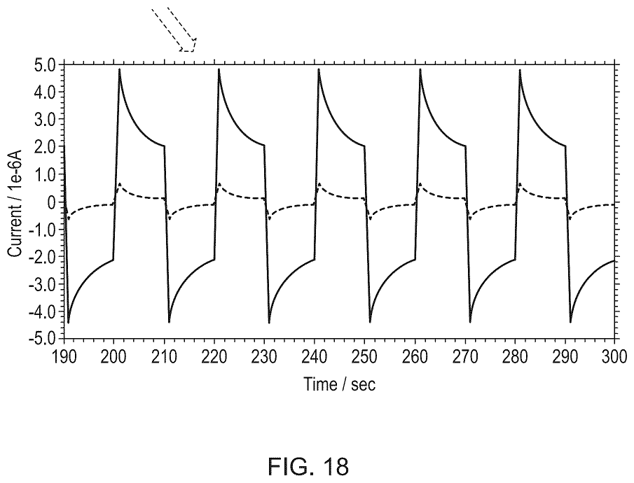

The sensors used in Example 4 (both the sensors with and without the hydrogel layer) were tested this time using chronoamperometry. The sensors were tested in a fluid comprising 20 mM of tris buffer, 140 mM NaCl and 6 mM KCl. Potential steps of +200 mV and −200 mV were used. Each potential step lasted only 10 seconds. Graphs showing the current arising from the flow of potassium ions into and out of the sensors on application of the potential pulses are depicted in FIGS. 17 and 18. It can be seen that for both sensors with and without the hydrogel, the currents obtained from the negative and positive potential pulses are nearly symmetrical. This is indicative of nearly full recovery of AgCl on the electrode and ejection of all potassium ions from the potassium selective transport layer on application of the positive potential pulse that had previously migrated into the potassium selective transport layer on application of the negative potential pulse. The observed currents are higher for the sensor comprising the hydrogel layer, but for both sensors, the current generated from the positive and negative potential pulses is almost equal (symmetrical). The results indicate that where shorter potential step duration chronoamperometric methods are used, both sensors with and without the presence of the hydrogel layer have sufficiently extended sensor lifetime and are thus suitable for use in in vivo methods of potassium detection in biological fluids. As shown in FIGS. 17 and 18, for both sensors, the current generated from the positive and negative pulses is similar and the graphs are symmetrical (in contrast to e.g., FIG. 14 where the current generated by the positive and negative potential steps is not equal indicating poor sensor lifetime).

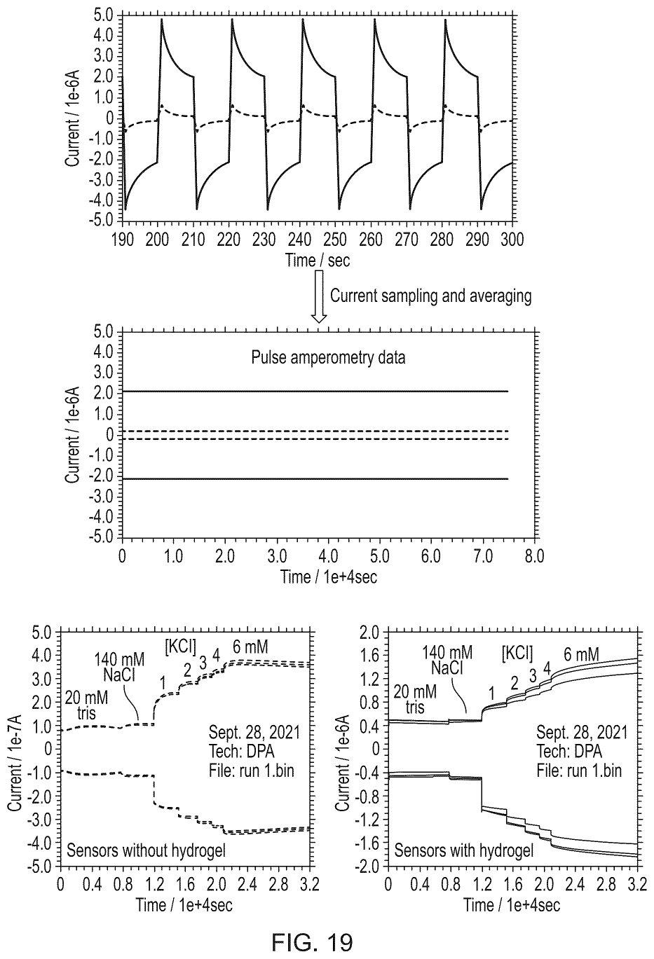

For the current shown in FIG. 18, pulse amperometry was used to sample the current generated over the latter half of the duration of each potential step. For example, for the potential step applied between 200 seconds and 210 seconds, the current between 205 seconds and 210 seconds was sampled by pulse amperometry. The average current observed over the latter 5 seconds of each 10 second potential step was sampled. The current generated during the latter half of each potential step is sampled because the current generated during the former half is not indicative of current generated due to potassium ions. The results of the pulse amperometry sampling for the data of FIG. 18 are shown in FIG. 19.

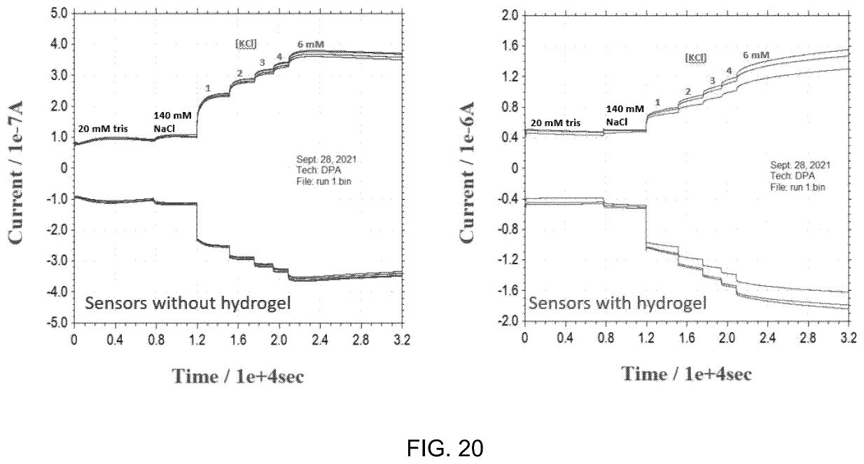

The sensors were tested again using the pulse amperometry methods discussed above at different potassium concentrations. The results of these experiments are shown in FIG. 20. It can be seen that for both sensors, at different potassium concentrations of from 1 to 6 mM, the current generated from the positive and negative potential steps was symmetrical and that similar current was generated on flow of the potassium ions into and out of the sensor. The results thus indicate that the sensors with and without the hydrogel both have a long sensor lifetime. A long sensor lifetime is preferable for use in in vivo potassium detection methods. The results also indicate that the sensors can be used to detect a variety of potassium concentrations found in vivo.

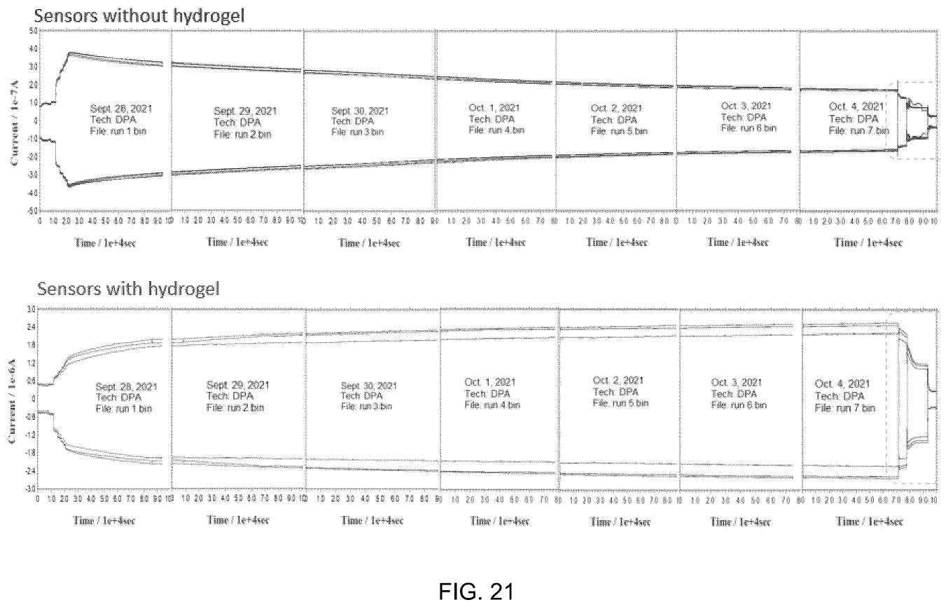

The sensors were tested on consecutive days, again, using pulse amperometry. The current generated from the respective positive and negative potential steps that were applied is shown in FIG. 21. In these experiments, potential steps of +200 mV and −200 mV were used. Each potential step lasted only 10 seconds. It can be seen in FIG. 21, that for the sensor that did not comprise a hydrogel layer, the current produced on application of the potential steps decreased slightly over time. This is indicative that the stability of the sensor was slightly decreasing over time. Over time, the transfer of potassium ions in and out of the potassium selective transport layer decreases, meaning that the functionality of the sensor is decreasing (although the sensor lifetime was still suitable for use for in vivo potassium detection in comparison to where different methods e.g., longer step duration chronoamperometry were used).

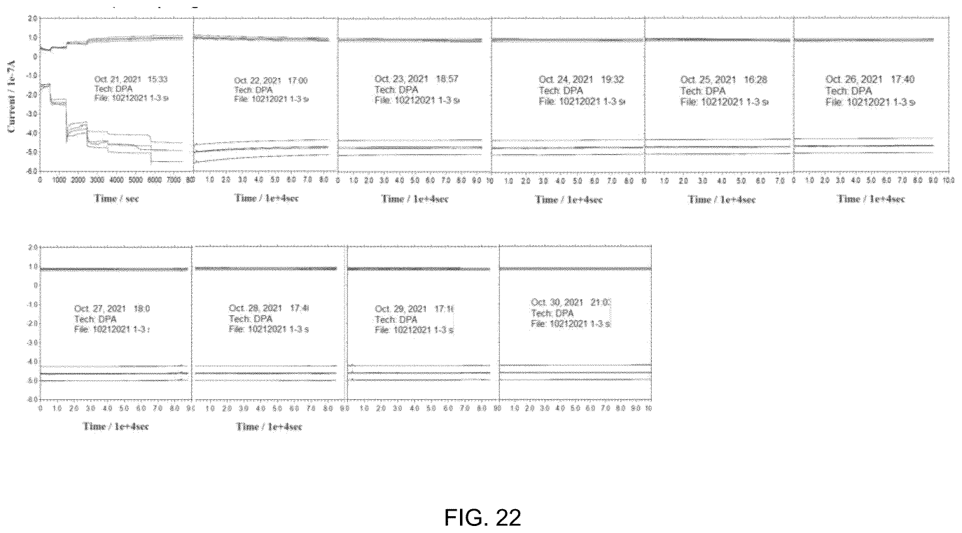

In an effort to increase sensor stability (and thus sensor lifetime), the pulse amperometry was carried out with 3 second positive potential steps and 1 second negative potential steps. Each potential step was 200 mV. Surprisingly, it was found that this change in pulse duration significantly increased sensor stability and lifetime. As can be seen in FIG. 22, even for sensors without the hydrogel layer, no decrease in the current generated from application of the positive and negative potential pulses was observed over 10 days.

1.-120. (canceled)

121. An analyte sensor comprising: a first working electrode and a potassium responsive active area disposed on at least a portion of an outer surface of the first working electrode; wherein the portion of the outer surface of the first working electrode comprises a transition metal ion in a salt or complex form, and optionally a transition metal; and wherein the potassium responsive active area comprises:
(i) a potassium selective transport layer forming an outer surface of the potassium responsive active area, wherein the potassium selective transport layer comprises a polymer, a potassium ionophore, a plasticizer, and an electrolyte; and
(ii) an electrolyte gel layer disposed between the potassium selective transport layer and the portion of the outer surface of the first working electrode.

122. The sensor of claim 121, wherein the polymer of the potassium selective transport layer comprises polyvinylchloride (PVC).

123. The sensor of claim 121, wherein the potassium ionophore of the potassium selective transport layer comprises valinomycin, Gramicidin A, laidlomycin, lasalocid, maduramicin, monensin, Narasin, Nigericin, Nonactin, Nystatin, Salinomycin, a crown ether, a cryptand, any derivative or conjugate thereof, or any combination thereof.

124. The sensor of claim 121, wherein the polymer of the potassium selective transport layer comprises polyvinylchloride (PVC) and wherein the plasticizer comprises a PVC plasticizer.

125. The sensor of claim 121, wherein the plasticizer comprises a phthalate compound, an adipate compound, an adipinate compound, a glutarate compound, a sebacate compound, a phosphate compound, a trimellitate compound, an epoxy compound, or a combination thereof; for example, wherein the plasticizer comprises 2-nitrophenyloctylether (NPOE).

126. The sensor of claim 121, wherein the electrolyte comprises a hydrophobic electrolyte.

127. The sensor of claim 121, wherein the electrolyte gel layer comprises an electrolyte hydrogel layer comprising an aqueous electrolyte solution and a crosslinked hydrophilic polymer.

128. The sensor of claim 127, wherein the crosslinked hydrophilic polymer comprises crosslinked polyvinyl alcohol, crosslinked polyethylene glycol, a crosslinked acrylate polymer, a crosslinked acrylamide polymer, a crosslinked hyaluronic polymer, a crosslinked chitosan, a crosslinked heparin, a crosslinked alginate, or a crosslinked fibrin.

129. The sensor of claim 121, wherein the sensor further comprises a support layer on which the first working electrode is disposed.

130. The sensor of claim 121, wherein the portion of the first working electrode comprises an osmium (III) ion, an osmium (II) ion, or a combination thereof.

131. The sensor of claim 121, wherein (i) the potassium selective transport layer comprises PVC, valinomycin, tetraoctylammonium tetrakis(pentafluorophenyl)borate (TOATB) and 2-nitrophenyloctylether (NPOE); and (ii) the electrolyte gel layer comprises an electrolyte hydrogel layer comprising an aqueous electrolyte solution of a Group 1 metal halide salt and crosslinked N, N-dimethylacrylamide.

132. The sensor of claim 121, wherein the potassium selective transport layer has a thickness of from 1 to 200 μm; the electrolyte gel layer has a thickness of from 1 to 50 μm; and/or the first working electrode layer has a thickness of from 1 nm to 20 μm.

133. The sensor of claim 121, wherein the sensor further comprises a layer of dielectric material disposed on a second portion of the outer surface of the first working electrode that the potassium responsive active area is not disposed on.

134. A method of detecting potassium ions in a fluid comprising:
(a) providing an analyte sensor comprising: a first working electrode and a potassium responsive active area disposed on at least a portion of an outer surface of the first working electrode; wherein the portion of the outer surface of the first working electrode comprises a transition metal ion in a salt or complex form, and optionally a transition metal; and wherein the potassium responsive active area comprises:
(i) a potassium selective transport layer forming an outer surface of the potassium responsive active area, wherein the potassium selective transport layer comprises a polymer, a potassium ionophore, a plasticizer, and an electrolyte; and
(ii) an electrolyte gel layer disposed between the potassium selective transport layer and the portion of the outer surface of the first working electrode;
(b) applying a potential to the first working electrode; and
(c) obtaining a signal indicative of oxidation and/or reduction of the transition metal ion in a salt or complex form, and optionally transition metal of the first working electrode; wherein the signal is indicative of the concentration of potassium ions in the fluid; and
(d) determining the concentration of potassium ions in the fluid from the signal obtained in step (c).

135. The method of claim 134, wherein the fluid comprises a biological fluid.

136. The method of claim 135, wherein the biological fluid comprises dermal fluid, interstitial fluid, plasma, blood, lymph, synovial fluid, cerebrospinal fluid, saliva, bronchoalveolar lavage or amniotic fluid.

137. The method of claim 134, wherein the sensor is located in vivo; and wherein the method comprises determining the concentration of potassium ions in an in vivo biological fluid.

138. The method of claim 134, wherein the method of detecting comprises a voltametric method; wherein step (b) comprises applying a varying potential to the first working electrode; and wherein the signal of step (c) comprises a peak current measurement.

139. The method of claim 134, wherein the method of detecting comprises an amperometric method; wherein step (b) comprises applying a constant potential to the first working electrode; and wherein step (c) comprises measuring the resultant current.

140. A method of detecting potassium ions in a fluid comprising:
(a) providing an analyte sensor comprising: a first working electrode and a potassium responsive active area disposed on at least a portion of an outer surface of the first working electrode; wherein the portion of the outer surface of the first working electrode comprises a transition metal ion in a salt or complex form, and optionally a transition metal; and wherein the potassium responsive active area comprises:
a potassium selective transport layer forming an outer surface of the potassium responsive active area, wherein the potassium selective transport layer comprises a polymer, a potassium ionophore, a plasticizer, and an electrolyte; and
(b) applying a potential to the first working electrode; and
(c) obtaining a signal indicative of oxidation and/or reduction of the transition metal ion in a salt or complex form, and optionally transition metal of the first working electrode; wherein the signal is indicative of the concentration of potassium ions in the fluid; and
(d) determining the concentration of potassium ions in the fluid from the signal obtained in step (c);
wherein steps (b) and (c) comprise using chronoamperometry to obtain the signal indicative of the concentration of potassium ions in the fluid, wherein the chronoamperometry comprises the successive application of first and second potentials; wherein the first potential is applied for a duration of from 10 milliseconds to 60 seconds, and wherein the second potential is applied for a duration of from 10 milliseconds to 60 seconds; and wherein the first potential is different from the second potential.
